(19) United States
(12) Patent Application Publication
(10) Pub. No.: US 2022/0064850 A1
(43) Pub. Date: Mar. 3, 2022

(54) NONWOVEN FABRIC HAVING IMPROVED FLUID MANAGEMENT PROPERTIES

(71) Applicant: Fitesa Simpsonville, Inc., Simpsonville, SC (US)

(72) Inventors: Rene Ruschel, Greer, SC (US); Fabio Baldissera, Simpsonville, SC (US)

(73) Assignee: Fitesa Simpsonville, Inc., Simpsonville, SC (US)

(21) Appl. No.: 17/411,356

(22) Filed: Aug. 25, 2021

Related U.S. Application Data
(60) Provisional application No. 63/074,417, filed on Sep. 3, 2020.

(51) Int. Cl. D06M 15/15; D04H 1/4291; D04H 1/435; D04H 1/724; D06B 3/10

(52) U.S. Cl. CPC D06M 15/15; D04H 1/4291; D06B 3/10; D04H 1/724; D04H 1/435

(57) ABSTRACT

A nonwoven fabric having improved fluid management properties is provided. The fabric is formed of a plurality of the fibers that include at least an aliphatic polyester polymer. The aliphatic polyester polymer defines at least a portion of an outer surface of the fibers, and includes a natural-based finish composition that is adhered thereto.

### CROSS-REFERENCE TO RELATED APPLICATIONS

This application claims priority to U.S. Provisional Application No. 63/074,417, filed Sep. 3, 2020, the entire disclosure of which is hereby incorporated herein by reference.

### FIELD

The presently-disclosed invention relates generally to nonwoven fabrics, and more particularly to nonwoven fabrics having improved fluid management properties.

### BACKGROUND

Nonwoven fabrics are used in a variety of applications such as garments, disposable medical products, and absorbent articles such as diapers and personal hygiene products, among others. New products being developed for these applications have demanding performance requirements, including comfort, conformability to the body, freedom of body movement, good softness and drape, adequate tensile strength and durability, and resistance to surface abrasion, pilling or fuzzing. Accordingly, the nonwoven fabrics which are used in these types of products must be engineered to meet these performance requirements.

In some absorbent articles, it may also be desirable for the nonwoven fabrics to facilitate rapid movement of fluids away from the skin of the wearer. For example, many absorbent articles include a topsheet and/or other fluid acquisition/distribution layers that transport fluids away from the wearer into an absorbent core of the article. However, many nonwoven fabrics for use in absorbent articles are prepared from materials that are inherently hydrophobic and therefore tend to repel water. Nonwoven fabrics made of such hydrophobic materials do not readily transport fluids away from the wearer. This is undesirable because it may result in leakage of fluids and discomfort to the wearer. To address this problem, the nonwoven fabric may be treated with finish compositions to help improve the hydrophilicity of the fabric and thereby facilitate the transport of fluids away from the skin of the wearer and into the absorbent core.

Despite significant efforts in developing nonwoven fabrics, there is still a need for products exhibiting improvements in fluid transport without sacrificing other beneficial properties such as tensile strength and elongation.

### SUMMARY

One or more embodiments of the invention may provide a nonwoven fabric having desirable properties with respect to fluid transport, comfort, softness, and drape while maintaining good mechanical properties, such as tensile strength and elongation.

Polyolefins, such as polypropylene, are used in a wide variety of nonwoven fabric applications due to their desirable mechanical properties, ease of manufacture, and cost, among other reasons. However, polyolefins are generally hydrophobic and therefore tend to repel water. Various finishing compositions have been developed to improve the hydrophilicity of such nonwoven fabrics. Examples of such hydrophilic compositions include synthetic surfactants that are applied to the surface of the fabrics. Synthetic surfactants have the potential to irritate the skin of the wearer, and may also be prone to be removed from the surface of the fabric during use.

Accordingly, there is a growing interest to replace synthetic surfactants with bio-based materials that are derived from renewable resources. That is, compositions that are obtained from natural-based materials. However, such natural-based compositions do not always readily adhere to the surface of the hydrophobic fibers making it difficult to prepare nonwoven fabrics having the desired hydrophilicity. Current methods of applying natural-based compositions to polyolefin fibers are generally complex and cannot readily be done at commercial processing speeds. For example, it has generally been found that to obtain enough of the natural-based composition on the fibers, the fibers must be exposed to a solution containing the natural-based composition for extended periods of time.

Certain embodiments of the present invention provide nonwoven fabrics having improved fluid management properties. In particular, embodiments of the invention are directed to nonwoven fabrics comprising fibers having an aliphatic polyester component to which a natural-based finish composition has been applied to the surface of the fabric. The inventors have found that natural-based finish compositions more readily adhere to aliphatic polyesters.

Advantageously, nonwoven fabrics in accordance with certain embodiments of the invention comprise a plurality of multicomponent fibers that are bonded to each other to form a coherent web, wherein the fibers have at least two distinct polymer components. The first polymer component comprises an aliphatic polyester polymer that defines at least a portion of an outer surface of the fibers, and the second polymer component comprises a polyolefin polymer, such as polypropylene. A natural-based composition is disposed on at least a portion of the outer surface of the fibers.

The inventors have discovered that natural-based compositions in accordance with embodiments of the invention may be more readily applied to surfaces of the fibers comprising the aliphatic polyester polymer. As a consequence, nonwoven fabrics having a natural-based finish composition may be prepared at viable commercial speeds without requiring complex steps for applying the composition to the surface of the fibers.

In certain embodiments, a nonwoven fabric comprising a plurality of fibers that are bonded to each other to form a coherent web in which the fibers comprise an aliphatic polyester polymer defining at least a portion of an outer surface of the fibers, and wherein a natural-based finish composition is disposed on at least a portion of the outer surface of the fibers is provided.

In some embodiments, the fibers comprise multicomponent fibers having a bicomponent configuration, in which a first polymer component comprises the aliphatic polyester polymer and defines a sheath of the fibers, and a second polymer component comprises a polyolefin polymer defining a core of the fibers.

In one embodiment, the aliphatic polyester polymer comprises polylactic acid, and the polyolefin polymer comprises polypropylene polymer, and in certain embodiments, the fibers comprise a bio-based aliphatic polyester polymer.

In certain embodiments, fibers have a sheath core configuration, and the bio-based aliphatic polyester polymer defines the sheath, and a second polymer component comprising a bio-based polymer is the core.

In one embodiment, a nonwoven fabric comprising a plurality of multicomponent fibers that are bonded to each other to form a coherent web is provided in which the fibers have at least two distinct polymer components and wherein the first polymer component comprises an aliphatic polyester polymer defining at least a portion of an outer surface of the fibers, and the second polymer component comprising a polyolefin polymer, and wherein a natural-based finish composition is disposed on at least a portion of the outer surface of the fibers.

In one embodiment, the fibers have a bicomponent configuration, and the first polymer component defines a sheath, and the second polymer component defines a core of the fiber. In some embodiments, the fibers have a side-by-side configuration.

In some embodiments, the aliphatic polyester polymer comprises polylactic acid, and the polyolefin polymer comprises polypropylene polymer.

In some embodiments, the natural-based finish composition comprises a plant or animal based protein, such as a milk-based protein or a soy protein isolate.

In some embodiments, the natural-based finish composition comprises an aqueous solution, suspension, or dispersion.

In one embodiment, the natural-based finish composition is present as a dried add-on amount ranging from about 0.2 to 2 weight percent, based on the total weight of the nonwoven fabric. In certain embodiments, the dried natural-based finish composition is present in an amount ranging from about 0.5 to 1.0 weight percent, based on the total weight of the nonwoven fabric.

In some embodiments, a treated nonwoven fabric in accordance with embodiments of the present invention exhibits a water droplet contact angle of less than 110 degrees, such as a water droplet contact angle of less than 105 degrees. In one embodiment, the treated nonwoven fabric exhibits a water droplet contact angle that is from about 95 to 105 degrees.

In certain embodiments, the nonwoven fabric treated with the natural-based finish composition exhibits a decrease of at least 10% in contact angle in comparison to the identical nonwoven fabric that has not been treated with the natural-based composition. In one embodiment the nonwoven fabric treated with the natural-based finish composition exhibits a decrease of at least 15% in contact angle in comparison to the identical nonwoven fabric that has not been treated with the natural-based composition, and in particular, a surface of the nonwoven fabric treated with the natural-based finish composition exhibits a decrease of at least 20% in contact angle in comparison to the identical nonwoven fabric that has not been treated with the natural-based composition.

In certain embodiments, a treated nonwoven fabric exhibits the following: a fluid strikethrough after a first insult of less than 5 seconds; a rewet of less than 1 gram; and a run-off of less than 5%.

In a further embodiment, the treated nonwoven fabric exhibits the following: a fluid strikethrough after a first insult of less than 2 seconds; a rewet of less than 0.5 grams; and a run-off of less than 1%.

In still further embodiments, the treated nonwoven fabric exhibits the following: a fluid strikethrough after a first insult of less than 1.75 seconds; a rewet of less than 0.2 grams; and a run-off of less than 0.5%.

In some embodiments, the polyolefin comprises a Zeigler-Natta catalyzed polypropylene or a metallocene catalyzed polypropylene, or a blend thereof.

In one embodiment, the polyolefin component comprises a polypropylene having a melt flow rate (MFR) that is from about 10 to 100 g/10 min, and in particular, from about 20 to 40 g/10 min, and more particularly, from about 22 to 38 g/10 min.

In some embodiments, the nonwoven fabric has a basis weight from about 7 grams per square meter (gsm) to about 150 gsm, and in particular, from about 11 gsm to about 30 gsm, such as from about 15 gsm to about 25 gsm.

In some embodiments, the nonwoven fabric is a spunbond fabric.

In some embodiments, the fibers have a linear mass density from about 0.8 dtex to about 3 dtex, and in particular from about 1 dtex to about 2.5 dtex, and more particularly, from about 1.2 dtex to about 2 dtex.

In some embodiments, the nonwoven fabric a percent area of an area bonded is from about 5 to 30%.

The inventive nonwoven fabric may be bonded with a CD rod bond pattern or an oval-elliptical bond pattern.

In some embodiments, the nonwoven fabric is part of an absorbent article, such as a diaper or feminine hygiene product.

The nonwoven fabric may be used to prepare a composite sheet material, such as a composite sheet comprising a meltblown layer. In one embodiment, the composite sheet comprises a meltblown layer is sandwiched between two spunbond layers, and wherein at least one of the spunbond layer is in accordance with a nonwoven fabric in accordance with embodiments of the invention.

In yet further aspects of the invention, a bio-based nonwoven fabric is provided. In these embodiments, the bio-based nonwoven fabric comprises a plurality of fibers that are bonded to each other to form a coherent web, and wherein the fibers comprising a bio-based aliphatic polyester polymer and having a natural-based finish composition disposed on at least a portion of the outer surfaces of the fibers.

In some embodiments, the bio-based nonwoven fabric may include fibers that are multicomponent or monocomponent.

In one embodiment of the bio-based nonwoven fabric, the fibers have a sheath core configuration wherein a first polymer component comprising an aliphatic polyester is the sheath, and a second polymer component comprising a bio-based polyethylene is the core.

In some embodiments of the bio-based nonwoven fabric, the fibers have a sheath core configuration, and wherein a first bio-based aliphatic polyester defines the sheath, and a second bio-based aliphatic polyester defines the core.

In some embodiments of the bio-based nonwoven fabric, the fibers are multicomponent and include first and second bio-based aliphatic polyesters that each comprise PLA.

In certain embodiments of the bio-based nonwoven fabric, the fabric includes a first bio-based aliphatic polyester comprising a first PLA resin having a first crystallinity and the second bio-based aliphatic polyester comprising a second PLA resin having a crystallinity different than said first crystallinity.

In some embodiments of the bio-based nonwoven fabric, the fibers of the nonwoven fabric include a first bio-based aliphatic polyester comprising a first PLA resin, and a second bio-based polyester comprising PBS.

In some embodiments of the bio-based nonwoven fabric, the fibers have a sheath core configuration wherein a first polymer component comprising an aliphatic polyester is the sheath, and a second polymer component is the core, and is selected from the group consisting of PBS, PHA, thermoplastic celluloses, thermoplastic starches, cellulose acetate, and combinations thereof.

In one embodiment of the bio-based nonwoven fabric, the fibers have a sheath/core configuration wherein a first polymer component defining the sheath is PBS, and second polymer component defining the core comprises a PLA.

In some embodiments of the bio-based nonwoven fabric, the natural-based finish composition comprises a plant or animal-based protein, and in particular, a soy protein isolate.

In certain embodiments of the bio-based nonwoven fabric, the natural-based finish composition comprises an aqueous solution or dispersion.

In some embodiments of the bio-based nonwoven fabric, wherein the dried natural-based finish composition is present in an amount ranging from about 0.2 to 2 weight percent, based on the total weight of the nonwoven fabric, and in particular, from about 0.5 to 1.0 weight percent, based on the total weight of the nonwoven fabric.

In some embodiments of the bio-based nonwoven fabric, the treated nonwoven fabric exhibits a water droplet contact angle of less than 125 degrees, such as a water droplet contact angle of less than 120 degrees, less than 115 degrees, or less than 110 degrees.

In one embodiment of the bio-based nonwoven fabric, the treated nonwoven fabric exhibits a water droplet contact angle that is from about 95 to 105 degrees.

In some embodiments of the bio-based nonwoven fabric the nonwoven fabric treated with the natural-based finish composition exhibits a decrease of at least 10% in contact angle in comparison to the identical nonwoven fabric that has not been treated with the natural-based composition.

In some embodiments of the bio-based nonwoven fabric, wherein the nonwoven fabric treated with the natural-based finish composition exhibits a decrease of at least 15% in contact angle in comparison to the identical nonwoven fabric that has not been treated with the natural-based composition.

In some embodiments of the bio-based nonwoven fabric, the nonwoven fabric treated with the natural-based finish composition exhibits a decrease of at least 20% in contact angle in comparison to the identical nonwoven fabric that has not been treated with the natural-based composition.

In some embodiments of the bio-based nonwoven fabric, the nonwoven fabric exhibits the following: a fluid strikethrough after a first insult of less than 5 seconds; a rewet of less than 1 gram; and a run-off of less than 5%.

In certain embodiments of the bio-based nonwoven fabric, the nonwoven fabric exhibits the following: a fluid strikethrough after a first insult of less than 2 seconds; a rewet of less than 0.5 grams; and a run-off of less than 1%.

In still further embodiments of the bio-based nonwoven fabric, the nonwoven fabric exhibits the following: a fluid strikethrough after a first insult of less than 1.75 seconds; a rewet of less than 0.2 grams; and a run-off of less than 0.5%.

Embodiments of the invention also include use of the nonwoven fabric in the manufacture of an absorbent article.

In one aspect, an absorbent article comprising a nonwoven fabric comprising a plurality of multicomponent fibers that are bonded to each other to form a coherent web is provided in which the fibers have at least two distinct polymer components, and wherein the first polymer component comprises polylactic acid polymer and defines at least a portion of an outer surface of the fibers, and the second polymer component comprising a polypropylene polymer, and wherein a natural-based finish composition is disposed on at least a portion of the outer surfaces of the fibers.

In some embodiments, the absorbent article is selected from the group consisting of wipes, diapers, adult incontinence products, and feminine hygiene products.

A further aspect is directed to providing an apparatus for preparing a nonwoven fabric in accordance with embodiments of the invention. In one such embodiment, the apparatus includes a supply source of a nonwoven fabric in which the nonwoven fabric comprises a plurality of fibers comprised of an aliphatic polyester polymer, the aliphatic polyester polymer being present on at least a portion of surfaces of the fibers; a source of heated fluid; a source of natural-based finish agent; a mixing tank in fluid communication with the source of heated fluid and source of natural-based finish agent, the mixing tank configured and arranged to mix the heated fluid and natural-based finish agent to produce a stream of heated composition comprising the natural-based agent; a heat exchanger in fluid communication for cooling the stream of heated composition comprising the natural-based agent to produce a stream of cooled composition comprising the natural-based agent; an application tank in fluid communication with the heat exchanger, the application tank configured and arranged to apply the cooled composition comprising the natural-based agent to fibers of the nonwoven fabric; and a dryer for removing fluid from the nonwoven fabric that has been treated with the composition comprising the natural-based agent.

In certain embodiments, the supply source of the nonwoven fabric comprises a spunbond fabric.

In one embodiment of the apparatus, the aliphatic polyester polymer comprises PLA.

In one embodiment of the apparatus, the fibers comprise a plurality of multicomponent fibers that are bonded to each other to form a coherent web, the fibers having at least two distinct polymer components wherein the first polymer component comprises an aliphatic polyester polymer and defines at least a portion of an outer surface of the fibers, and the second polymer component comprising a polyolefin polymer. In some embodiments, the first polymer component comprises PLA and the second polymer component comprises polypropylene.

In a further aspect, method for preparing a nonwoven fabric comprising:

providing a nonwoven fabric, the nonwoven fabric comprising a plurality of fibers comprised of an aliphatic polyester polymer, the aliphatic polyester polymer being present on at least a portion of surfaces of the fibers;
heating a fluid;
adding a natural-based finish agent to the heated fluid;
mixing the natural-based finish agent and heated fluid to produce a stream of heated composition comprising the natural-based agent;
cooling the heated composition comprising the natural-based agent to produce a stream of cooled composition comprising the natural-based agent;
applying the cooled composition comprising the natural-based agent to fibers of the nonwoven fabric; and
drying the nonwoven fabric that has been treated with the composition comprising the natural-based agent.

In some embodiments of the method, the cooled composition comprising the natural-based agent is applied to a surface of the nonwoven fabric via a kiss roll.

In one embodiment, the method includes continuously applying the natural-based finish composition to the fabric.

In one embodiment, the natural-based agent is mixed with water to produce a stream of heated composition.

In certain embodiments of the method, the fibers have a bicomponent configuration comprising a first polymer component and a second polymer component, and wherein the first polymer component defines a sheath, and the second polymer component defines a core of the fiber. In some particular embodiments of the method, the first polymer component comprises a polymer selected from the group consisting of polylactic acid (PLA), polybutylene succinate (PBS), and mixtures thereof, and the second polymer comprises polypropylene.

In some embodiments of the method, the fibers of the nonwoven fabric are bio-based. In other embodiments of the method, the fibers have a bio-based polymer component and a synthetic polymer component.

In some embodiments of the method, the bio-based fibers are monocomponent.

In certain embodiments of the method, the fibers are monocomponent and are substantially comprised of bio-based polymers.

In certain embodiments of the method, the fibers of the nonwoven fabric have a sheath/core configuration, and the sheath comprises PLA, and the core is a polymer selected from the group consisting of PBS, PHA, thermoplastic celluloses, thermoplastic starches, cellulose acetate, and combinations thereof.

In certain embodiments of the method, the fibers of the nonwoven fabric have a sheath/core configuration, and the sheath comprises a PLA polymer, and the core comprise a PLA polymer.

In certain embodiments of the method, the fibers of the nonwoven fabric have a sheath/core configuration, and the sheath comprises a PBS polymer, and the core comprise a PLA polymer.

In certain embodiments of the method, the fibers of the nonwoven fabric have a sheath/core configuration, and the sheath comprises a PLA polymer, and the core comprise a bio-based polyethylene.

In certain embodiments of the method, the step of applying the cooled composition is performed at a line speed from about 50 to 1,200 meters/minute.

In certain embodiments of the method, step of cooling the heated composition comprises cooling the composition to a temperature less than about 40° C.

In certain embodiments of the method, the step of cooling the heated composition comprises cooling the composition to a temperature ranging from about 25 to 35° C.

In certain embodiments of the method, two separate streams of heated composition comprising the natural-based agent are produced and successively introduced into a heat exchanger to provide a continuous stream of the cooled composition comprising the natural-based agent to the application tank.

### BRIEF DESCRIPTION OF THE SEVERAL DRAWINGS

Having thus described the invention in general terms, reference will now be made to the accompanying drawings, which are not necessarily drawn to scale, and wherein:

FIG. 1 illustrates a system for preparing nonwoven fabrics in accordance with embodiments of the present invention;

FIG. 2 illustrates a system for preparing and applying a natural-based finish composition to the surface of a nonwoven fabric in accordance with embodiments of the present invention;

FIGS. 3A-3D illustrate various composite fabric structures that in accordance with one or more embodiments of the present invention;

FIGS. 4A and 4B illustrate an absorbent article in accordance with one or more embodiments of the present invention; and

FIG. 5 illustrates an absorbent article in accordance with one or more embodiments of the present invention.

### DETAILED DESCRIPTION

The invention now will be described more fully hereinafter with reference to the accompanying drawings, in which some, but not all embodiments of the inventions are shown. Indeed, the invention may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

The terms “first,” “second,” and the like, “primary,” “exemplary,” “secondary,” and the like, do not denote any order, quantity, or importance, but rather are used to distinguish one element from another. Further, the terms “a,” “an,” and “the” do not denote a limitation of quantity, but rather denote the presence of “at least one” of the referenced item.

Each embodiment disclosed herein is contemplated as being applicable to each of the other disclosed embodiments. All combinations and sub-combinations of the various elements described herein are within the scope of the invention.

It is understood that where a parameter range is provided, all integers within that range, and tenths and hundredths thereof, are also provided by the invention. For example, “5-10%” includes 5%, 6%, 7%, 8%, 9%, and 10%; 5.0%, 5.1%, 5.2% . . . 9.8%, 9.9%, and 10.0%; and 5.00%, 5.01%, 5.02% . . . 9.98%, 9.99%, and 10.00%.

As used herein, the terms “about,” “approximately,” and “substantially” in the context of a numerical value or range means ±10% of the numerical value or range recited or claimed, and in particular, encompasses values within a standard margin of error of measurement (e.g., SEM) of a stated value or variations ±0.5%, 1%, 5%, or 10% from a specified value.

For the purposes of the present application, the following terms shall have the following meanings:

The term “fiber” can refer to a fiber of finite length or a filament of infinite length.

As used herein, the term “monocomponent” refers to fibers formed from one polymer or formed from a single blend of polymers. Of course, this does not exclude fibers to which additives have been added for color, anti-static properties, lubrication, hydrophilicity, liquid repellency, etc.

As used herein, the term “multicomponent” refers to fibers formed from at least two polymers (e.g., bicomponent fibers) that are extruded from separate extruders. The at least two polymers can each independently be the same or different from each other, or be a blend of polymers. The polymers are arranged in substantially constantly positioned distinct zones across the cross-section of the fibers. The components may be arranged in any desired configuration, such as sheath-core, side-by-side, pie, island-in-the-sea, and so forth. Various methods for forming multicomponent fibers are described in U.S. Pat. No. 4,789,592 to Taniguchi et al. and U.S. Pat. No. 5,336,552 to Strack et al., U.S. Pat. No. 5,108,820 to Kaneko, et al., U.S. Pat. No. 4,795,668 to Kruege, et al., U.S. Pat. No. 5,382,400 to Pike, et al., U.S. Pat. No. 5,336,552 to Strack, et al., and U.S. Pat. No. 6,200,669 to Marmon, et al., which are incorporated herein in their entirety by reference. Multicomponent fibers having various irregular shapes may also be formed, such as described in U.S. Pat. No. 5,277,976 to Hogle, et al., U.S. Pat. No. 5,162,074 to Hills, U.S. Pat. No. 5,466,410 to Hills, U.S. Pat. No. 5,069,970 to Largman, et al., and U.S. Pat. No. 5,057,368 to Largman, et al., which are incorporated herein in their entirety by reference.

As used herein the terms “nonwoven,” “nonwoven web” and “nonwoven fabric” refer to a structure or a web of material which has been formed without use of weaving or knitting processes to produce a structure of individual fibers or threads which are intermeshed, but not in an identifiable, repeating manner. Nonwoven webs have been, in the past, formed by a variety of conventional processes such as, for example, meltblown processes, spunbond processes, and staple fiber carding processes.

As used herein, the term “meltblown” refers to a process in which fibers are formed by extruding a molten thermoplastic material through a plurality of fine, usually circular, die capillaries into a high velocity gas (e.g. air) stream which attenuates the molten thermoplastic material and forms fibers, which can be to microfiber diameter. Thereafter, the meltblown fibers are carried by the gas stream and are deposited on a collecting surface to form a web of random meltblown fibers. Such a process is disclosed, for example, in U.S. Pat. No. 3,849,241 to Buntin et al.

As used herein, the term “machine direction” or “MD” refers to the direction of travel of the nonwoven web during manufacturing.

As used herein, the term “cross direction” or “CD” refers to a direction that is perpendicular to the machine direction and extends laterally across the width of the nonwoven web.

As used herein, the term “natural-based finish composition” refers to a composition derived from naturally sourced materials, and that is applied to the nonwoven fabric to render the nonwoven fabric more hydrophilic in comparison to the untreated fabric. Such naturally sourced materials may include water, minerals, and ingredients of mineral origins that are physically or chemically processed from plant or animal sources. Preferably, natural-based finish compositions are substantially free of synthetic materials, such as materials derived from a petrochemical source.

As used herein, and unless indicated to the contrary, the term “molecular weight” refers to the weight average molecular weight (Mw), and is expressed in grams/mol. The weight average molecular weight can be determined using commonly known techniques, such as gel permeation chromatography (GPC).

As used herein, the term “spunbond” refers to a process involving extruding a molten thermoplastic material as filaments from a plurality of fine, usually circular, capillaries of a spinneret, with the filaments then being attenuated and drawn mechanically or pneumatically. The filaments are deposited on a collecting surface to form a web of randomly arranged substantially continuous filaments which can thereafter be bonded together to form a coherent nonwoven fabric. The production of spunbond non-woven webs is illustrated in patents such as, for example, U.S. Pat. Nos. 3,338,992; 3,692,613, 3,802,817; 4,405,297 and 5,665,300. In general, these spunbond processes include extruding the filaments from a spinneret, quenching the filaments with a flow of air to hasten the solidification of the molten filaments, attenuating the filaments by applying a draw tension, either by pneumatically entraining the filaments in an air stream or mechanically by wrapping them around mechanical draw rolls, depositing the drawn filaments onto a foraminous collection surface to form a web, and bonding the web of loose filaments into a nonwoven fabric. The bonding can be any thermal or chemical bonding treatment, with thermal point bonding being typical.

As used herein “thermal point bonding” involves passing a material such as one or more webs of fibers to be bonded between a heated calender roll and an anvil roll. The calender roll is typically patterned so that the fabric is bonded in discrete point bond sites rather than being bonded across its entire surface.

As used herein the term “polymer” generally includes, but is not limited to, homopolymers, copolymers, such as, for example, block, graft, random and alternating copolymers, terpolymers, etc. and blends and modifications thereof. Furthermore, unless otherwise specifically limited, the term “polymer” shall include all possible geometrical configurations of the material, including isotactic, syndiotactic and random symmetries.

Nonwoven Fabric

I. Bicomponent Embodiment Having an Aliphatic Polyester

In certain embodiments, aspects of the invention are directed to nonwoven fabrics comprising multicomponent fibers that include an aliphatic polyester component and a polyolefin component, and in which a natural-based finish composition is present on at least a portion of the exterior surfaces of the fibers.

In one embodiment, the present invention provides a spunbond nonwoven fabric comprising a plurality of multicomponent fibers that are bonded to each other to form a coherent web.

Although, the present invention is generally discussed in the context of spunbond fabrics prepared from continuous filaments, it should be recognized that other nonwoven fabrics and fibers may be prepared in accordance of the invention including meltblown fibers and meltblown fabrics, coformed fabrics, bi-formed fabrics, staple fibers and carded fabric, wet-laid fabrics, and air-laid fabrics.

In one aspect, embodiments of the invention are directed to a nonwoven fabric comprising multicomponent filaments in which a first component comprises an aliphatic polyester component, and a second component comprises a polypropylene component. Advantageously, it has been discovered that natural-based finish compositions may be more readily applied to the surface of fibers comprising an aliphatic polyester, such as a polylactic acid (PLA) polymer, in comparison to polypropylene.

In one embodiment, the multicomponent fibers of the invention may include at least two polymer components arranged in structured domains across the cross section of the fiber. As is generally well known to those skilled in the art, polymer domains or components are arranged in substantially continuously positioned zones across the cross-section of the multicomponent fiber and extend continuously along the length of the multicomponent fiber. More than two components could be present in the multicomponent fiber. A preferred configuration is a sheath/core arrangement wherein a first component, the sheath, substantially surrounds a second component, the core. The resulting sheath/core bicomponent fiber may have a round or non-round cross-section. Other structured fiber configurations as known in the art can be used including side-by-side, segmented pie, islands-in-the-sea and tipped multilobal structures.

In a preferred embodiment, the fibers are bicomponent in which the aliphatic polyester component defines a sheath of the fiber, and the polypropylene component defines a core of the fiber. Generally, the weight percentage of the sheath to that of the core in the fibers may vary widely depending upon the desired properties of the nonwoven fabric. For example the weight ratio of the sheath to the core may vary between about 5:95 to 95:5, such as from about 10:90 to 90:10, and in particular from about 20:80 to 80:20. In a preferred embodiment, the weight ratio of the sheath to the core is about 25:75 to 35:65, with a weight ratio of about 20:80 to 50:50 being preferred.

Preferred sheath/core bicomponent fibers for use in making fabrics of this invention can have the higher melting component as the core and the lower melting component as the sheath. For example, the aliphatic polyester component could be used as the sheath and the core could be a higher melting polymer component comprising a polyolefin, such as polypropylene. Such a structure with an aliphatic polyester on the surface allows use of a reduced calender bonding temperature thus conserving energy during manufacture of the nonwoven web.

Aliphatic Polyester Component

Aliphatic polyesters useful in the present invention include homo- and copolymers of poly(hydroxyalkanoates), and homo- and copolymers of those aliphatic polyesters derived from the reaction product of one or more polyols with one or more polycarboxylic acids that are typically formed from the reaction product of one or more alkanediols with one or more alkanedicarboxylic acids (or acyl derivatives). Polyesters may further be derived from multifunctional polyols, e.g. glycerin, sorbitol, pentaerythritol, and combinations thereof, to form branched, star, and graft homo- and copolymers. Polyhydroxyalkanoates generally are formed from hydroxyacid monomeric units or derivatives thereof. These include, for example, polylactic acid, polyhydroxybutyrate, polyhydroxyvalerate, polycaprolactone and the like. Miscible and immiscible blends of aliphatic polyesters with one or more additional semicrystalline or amorphous polymers may also be used.

One useful class of aliphatic polyesters are poly(hydroxyalkanoates), derived by condensation or ring-opening polymerization of hydroxy acids, or derivatives thereof. Suitable poly(hydroxyalkanoates) may be represented by the formula: H(O—R—C(O)—)ₙOH where R is an alkylene moiety that may be linear or branched having 1 to 20 carbon atoms, preferably 1 to 12 carbon atoms optionally substituted by catenary (bonded to carbon atoms in a carbon chain) oxygen atoms; n is a number such that the ester is polymeric, and is preferably a number such that the molecular weight of the aliphatic polyester is at least 10,000, preferably at least 30,000, and most preferably at least 50,000 daltons. In certain embodiments, the molecular weight of the aliphatic polyester is typically less than 1,000,000, preferably less than 500,000, and most preferably less than 300,000 daltons. R may further comprise one or more caternary (i.e. in chain) ether oxygen atoms. Generally, the R group of the hydroxy acid is such that the pendant hydroxyl group is a primary or secondary hydroxyl group.

Useful poly(hydroxyalkanoates) include, for example, homo- and copolymers of poly(3-hydroxybutyrate), poly(4-hydroxybutyrate), poly(3-hydroxyvalerate), poly(lactic acid) (as known as polylactide), poly(3-hydroxypropanoate), poly(4-hydropentanoate), poly(3-hydroxypentanoate), poly(3-hydroxyhexanoate), poly(3-hydroxyheptanoate), poly(3-hydroxyoctanoate), polydioxanone, polycaprolactone, and polyglycolic acid (i.e. polyglycolide). Copolymers of two or more of the above hydroxy acids may also be used, for example, poly(3-hydroxybutyrate-co-3-hydroxyvalerate), poly(lactate-co-3-hydroxypropanoate), poly(glycolide-co-p-dioxanone), and poly(lactic acid-co-glycolic acid). Blends of two or more of the poly(hydroxyalkanoates) may also be used, as well as blends with one or more semicrystalline or amorphous polymers and/or copolymers.

The aliphatic polyester may be a block copolymer of poly(lactic acid-co-glycolic acid). Aliphatic polyesters useful in the inventive compositions may include homopolymers, random copolymers, block copolymers, star-branched random copolymers, star-branched block copolymers, dendritic copolymers, hyperbranched copolymers, graft copolymers, and combinations thereof.

Another useful class of aliphatic polyesters includes those aliphatic polyesters derived from the reaction product of one or more alkanediols with one or more alkanedicarboxylic acids (or acyl derivatives). Such polyesters have the general formula:

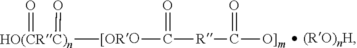

where R′ and R″ each represent an alkylene moiety that may be linear or branched having from 1 to 20 carbon atoms, preferably 1 to 12 carbon atoms, and m is a number such that the ester is polymeric, and is preferably a number such that the molecular weight of the aliphatic polyester is at least 10,000, preferably at least 30,000, and most preferably at least 50,000 daltons, but less than 1,000,000, preferably less than 500,000 and most preferably less than 300,000 daltons. Each n is independently 0 or 1. R′ and R″ may further comprise one or more caternary (i.e. in chain) ether oxygen atoms.

Examples of aliphatic polyesters include those homo- and copolymers derived from (a) one or more of the following diacids (or derivative thereof): succinic acid; adipic acid; 1,12 dicarboxydodecane; fumaric acid; glutartic acid; diglycolic acid; and maleic acid; and (b) one of more of the following diols: ethylene glycol; polyethylene glycol; 1,2-propane diol; 1,3-propanediol; 1,2-propanediol; 1,2-butanediol; 1,3-butanediol; 1,4-butanediol; 2,3-butanediol; 1,6-hexanediol; 1,2 alkane diols having 5 to 12 carbon atoms; diethylene glycol; polyethylene glycols having a molecular weight of 300 to 10,000 daltons, and preferably 400 to 8,000 daltons; propylene glycols having a molecular weight of 300 to 4000 daltons; block or random copolymers derived from ethylene oxide, propylene oxide, or butylene oxide; dipropylene glycol; and polypropylene glycol, and (c) optionally a small amount, i.e., 0.5-7.0 mole percent of a polyol with a functionality greater than two, such as glycerol, neopentyl glycol, and pentaerythritol.

Such polymers may include polybutylene succinate homopolymer, polybutylene adipate homopolymer, polybutyleneadipate-succinate copolymer, polyethylenesuccinate-adipate copolymer, polyethylene glycol succinate homopolymer and polyethylene adipate homopolymer.

Commercially available aliphatic polyesters include poly(lactide), poly(glycolide), poly(lactide-co-glycolide), poly(L-lactide-co-trimethylene carbonate), poly(dioxanone), poly(butylene succinate), and poly(butylene adipate).

The term “aliphatic polyester” covers—besides polyesters which are made from aliphatic and/or cycloaliphatic components exclusively also polyesters which contain besides aliphatic and/or cycloaliphatic units, aromatic units, as long as the polyester has substantial sustainable content.

In addition to PLA based resins, nonwoven fabrics in accordance with embodiments of the invention may include other polymers derived from an aliphatic component possessing one carboxylic acid group and one hydroxyl group, which are alternatively called polyhydroxyalkanoates (PHA). Examples thereof are polyhydroxybutyrate (PHB), poly-(hydroxybutyrate-co-hydroxyvaleterate) (PHBV), poly-(hydroxybutyrate-co-polyhydroxyhexanoate) (PHBH), polyglycolic acid (PGA), poly-(epsilon-caprolactione) (PCL) and preferably polylactic acid (PLA).

Examples of additional polymers that may be used in embodiments of the invention include polymers derived from a combination of an aliphatic component possessing two carboxylic acid groups with an aliphatic component possessing two hydroxyl groups, and are polyesters derived from aliphatic diols and from aliphatic dicarboxylic acids, such as polybutylene succinate (PB SU), polyethylene succinate (PESU), polybutylene adipate (PBA), polyethylene adipate (PEA), polytetramethy-lene adipate/terephthalate (PTMAT).

Useful aliphatic polyesters include those derived from semicrystalline polylactic acid. Poly(lactic acid) or polylactide (PLA) has lactic acid as its principle degradation product, which is commonly found in nature, is non-toxic and is widely used in the food, pharmaceutical and medical industries. The polymer may be prepared by ring-opening polymerization of the lactic acid dimer, lactide. Lactic acid is optically active and the dimer appears in four different forms: L,L-lactide, D,D-lactide, D,L-lactide (meso lactide) and a racemic mixture of L,L- and D,D-. By polymerizing these lactides as pure compounds or as blends, poly(lactide) polymers may be obtained having different stereochemistries and different physical properties, including crystallinity. The L,L- or D,D-lactide yields semicrystalline poly(lactide), while the poly(lactide) derived from the D,L-lactide is amorphous.

Generally, polylactic acid based polymers are prepared from dextrose, a source of sugar, derived from field corn. In North America corn is used since it is the most economical source of plant starch for ultimate conversion to sugar. However, it should be recognized that dextrose can be derived from sources other than corn. Sugar is converted to lactic acid or a lactic acid derivative via fermentation through the use of microorganisms. Lactic acid may then be polymerized to form PLA. In addition to corn, other agricultural based sugar sources may be used including rice, sugar beets, sugar cane, wheat, cellulosic materials, such as xylose recovered from wood pulping, and the like.

The polylactide preferably has a high enantiomeric ratio to maximize the intrinsic crystallinity of the polymer. The degree of crystallinity of a poly(lactic acid) is based on the regularity of the polymer backbone and the ability to crystallize with other polymer chains. If relatively small amounts of one enantiomer (such as D-) is copolymerized with the opposite enantiomer (such as L-) the polymer chain becomes irregularly shaped, and becomes less crystalline. For these reasons, when crystallinity is favored, it is desirable to have a poly(lactic acid) that is at least 85% of one isomer, at least 90% of one isomer, or at least 95% of one isomer in order to maximize the crystallinity.

In some embodiments, an approximately equimolar blend of D-polylactide and L-polylactide is also useful. This blend forms a unique crystal structure having a higher melting point (about 210° C.) than does either the D-poly(lactide) and L-(polylactide) alone (about.190° C.), and has improved thermal stability.

Copolymers, including block and random copolymers, of poly(lactic acid) with other aliphatic polyesters may also be used. Useful co-monomers include glycolide, beta-propiolactone, tetramethylglycolide, beta-butyrolactone, gamma-butyrolactone, pivalolactone, 2-hydroxybutyric acid, alpha-hydroxyisobutyric acid, alpha-hydroxyvaleric acid, alpha-hydroxyisovaleric acid, alpha-hydroxycaproic acid, alpha-hydroxyethylbutyric acid, alpha-hydroxyisocaproic acid, alpha-hydroxy-beta-methylvaleric acid, alpha-hydroxyoctanoic acid, alpha-hydroxydecanoic acid, alpha-hydroxymyristic acid, and alpha-hydroxystearic acid.

Blends of poly(lactic acid) and one or more other aliphatic polyesters, or one or more other polymers may also be used. Examples of useful blends include poly(lactic acid) and poly(vinyl alcohol), polyethylene glycol/polysuccinate, polyethylene oxide, polycaprolactone and polyglycolide.

In certain preferred embodiments, the aliphatic polyester component comprises a PLA based resin. A wide variety of different PLA resins may be used to prepare nonwoven fabrics in accordance with embodiments of the invention. The PLA resin should have proper molecular properties to be spun in spunbond processes. Examples of suitable include PLA resins are supplied from NatureWorks LLC, of Minnetonka, Minn. 55345 such as, grade 6752D, 6100D, and 6202D, which are believed to be produced as generally following the teaching of U.S. Pat. Nos. 5,525,706 and 6,807,973 both to Gruber et al. Other examples of suitable PLA resins may include L130, L175, and LX175, all from Corbion of Arkelsedijk 46, 4206 A C Gorinchem, the Netherlands.

In some embodiments, the inventive nonwoven fabrics may comprise sustainable polymer components of biodegradable products that are derived from an aliphatic component possessing one carboxylic acid group (or a polyester forming derivative thereof, such as an ester group) and one hydroxyl group (or a polyester forming derivative thereof, such as an ether group) or may be derived from a combination of an aliphatic component possessing two carboxylic acid groups (or a polyester forming derivative thereof, such as an ester group) with an aliphatic component possessing two hydroxyl groups (or a polyester forming derivative thereof, such as an ether group).

Polyolefin Component

In certain embodiments, the polyolefin component may comprise polyethylene, polypropylene, and copolymers and/or blends thereof. In a preferred embodiment, the polyolefin component comprises polypropylene.

A wide variety of polypropylenes may be used as the polyolefin component in the fibers. Suitable polypropylenes may be produced from any of the well-known processes, including metallocene and Ziegler-Natta catalyst systems.

Generally, the polypropylene used in the invention has a relatively high degree of crystallinity, such as above 70%, and has a melting point greater than about 130° C. without particular limitation. Examples thereof include a propylene homopolymer, a propylene random copolymer and a propylene block copolymer.

In one embodiment, the first polypropylene in the polypropylene component is a standard polypropylene used in the preparation of spunbond nonwoven fabrics. Such polypropylenes are typically characterized by melting points greater than 130° C.

In one embodiment, the polypropylene has a melting temperature that is greater than at least 150° C., greater than at least 155° C., greater than at least 160° C., greater than at least 165° C., greater than at least 170° C., and greater than at least 175° C. The melting temperature of polypropylene can be determined in accordance with ASTM D 3418.

Polypropylenes that may be used in embodiments of the invention typically have molecular weights greater than about 120,000 g/mol, and more typically, may have molecular weights ranging from about 150,000 to about 300,000 g/mol. In one embodiment, the first polypropylene may have molecular weights ranging from about 160,000 to about 250,000 g/mol, and in particular, from about 160,000 to about 180,000 g/mol.

In addition, polypropylenes that may be used have an MFR that is from about 10 to 100 g/10 min, and in particular, from about 20 to 40 g/10 min, with an MFR from about 22 to 38 g/10 min being somewhat more typical. Unless otherwise indicated MFR is measured in accordance with ASTM D-1238.

Examples of such polypropylenes may include those available from ExxonMobil, such as PP3155 (36 MFR g/10 min, density of 0.90 g/cm³, and Mw 172 k g/mol); PP3155E5 (36 MFR g/10 min, density of 0.90 g/cm³, and Mw 172 k g/mol); and ACHIEVE™ 3854 (24 MFR g/10 min, density of 0.90 g/cm³). Polypropylenes available from SABIC®, such as SABIC PP 511A (25 MFR g/10 min, density of 0.905 g/cm³), and polypropylenes available from Borealis, such as HG475FB (27 MFR g/10 min) may also be used.

In some embodiments, the polyolefin may comprise a polyethylene polymer. Various types of polyethylene polymers may be employed in the fibers of the present invention. As an example, a high density polyethylene, a branched (i.e., non-linear) low density polyethylene, or a linear low density polyethylene (LLDPE) can be utilized. Polyethylenes may be produced from any of the well-known processes, including metallocene and Ziegler-Natta catalyst systems. Generally, the polyethylene polymers that are conventionally used in the production of bicomponent fibers may be suitable for use in the present invention.

In one embodiment of the invention, the polyethylene component comprises a polyethylene having a density ranging from about 0.90 to 0.97 g/cm³ (ASTM D-792). In particular, preferred polyethyelenes have a density value ranging from 0.93 to 0.965 g/cm³, and more particularly from about 0.94 to 0.965 g/cm³. Examples of suitable polyethylenes included ASPUN™ 6834 (a polyethylene polymer resin having a melt index of 17 g/10 min (ISO 1133) and a density of 0.95 g/cm³ (ASTM D-792)), available from Dow Chemical Company, and HD6908.19 (a polyethylene resin supplied by ExxonMobil having a melt index in the range of 7.5 to 9 g/10 min (ISO 1133) and a density of 0.9610 to 0.9680 g/cm³ (ASTM D-792)).

LLDPE may also be used in some embodiments of the present invention. LLDPE is typically produced by a catalytic solution or fluid bed process under conditions established in the art. The resulting polymers are characterized by an essentially linear backbone. Density is controlled by the level of comonomer incorporated into the otherwise linear polymer backbone. Various alpha-olefins are typically copolymerized with ethylene in producing LLDPE. The alpha-olefins which preferably have four to eight carbon atoms, are present in the polymer in an amount up to about 10 percent by weight. The most typical comonomers are butene, hexene, 4-methyl-1-pentene, and octene. In general, LLDPE can be produced such that various density and melt index properties are obtained which make the polymer well suited for melt-spinning with polypropylene. Preferably, the LLDPE should have a melt index of greater than 10, and more preferably 15 or greater for spunbonded filaments. Particularly preferred are LLDPE polymers having a density of 0.90 to 0.97 g/cm³ and a melt index of greater than 25. Examples of suitable commercially available linear low density polyethylene polymers include those available from Dow Chemical Company, such as ASPUN™ Type 6811 (27 MFR g/10 min, density 0.923 g/cm³), ASPUN™ Type 6834 (17 MFR g/10 min, density of 0.95 g/cm³), ASPUN™ Type 6000 (30 MFR g/10 min, 0.955 g/cm³ density), ASPUN™ Type 6850 (30 MFR g/10 min, 0.955 g/cm³ density), Dow LLDPE 2500 (55 MFR g/10 min, 0.923 g/cm³ density), Dow LLDPE Type 6808A (36 MFR g/10 min, 0.940 g/cm³ density), and the Exact series of linear low density polyethylene polymers from Exxon Chemical Company, such as Exact 2003 (31 MFR g/10 min, density 0.921 g/cm³).

Natural-Based Composition

The natural-based finish composition is applied to the nonwoven fabric to improve the fluid management properties of the nonwoven fabric. As discussed previously, it has been discovered that natural-based compositions in accordance with embodiments of the invention adhere more readily to the surface of fibers comprising an aliphatic polyester, such as PLA. In this way, nonwoven fabrics can be prepared having improved hydrophilicity in which the natural-based composition is disposed on portions of exterior surfaces of the fibers that comprise an aliphatic polyester. This is particularly advantageous in embodiments of the invention in which the fibers comprise an aliphatic polyester sheath and a polyolefin core.

A wide variety of natural-based agents may be used in the preparation of the natural-based finish composition. In certain embodiments, the natural-based agent comprises proteins derived from plant and animal sources. Examples of animal derived proteins may include milk-based protein (whey protein and caseins protein) and egg protein. Examples of plant-based proteins may include pea protein, hemp protein, brown rice protein, chia seeds protein, alfalfa protein, flax seeds protein, quinoa protein, artichoke protein, and soy protein. The natural-based agent may also include combinations of the afore-mentioned proteins.

In a preferred embodiment, the natural-based agent comprises a water-soluble soy protein isolate (SPI). Examples of suitable SPIs that may be used in certain embodiments of the invention include Pro-Fam® 781 and Clarisoy® 100: both of which are 100% water-soluble soy protein isolate (SPI) commercially available from Archer Daniels Midland (ADM) and Burcon NutraScience Corporation.

In certain embodiments, the natural-based finish composition is applied to the fibers as an aqueous solution comprising the natural-based finish composition. In such embodiments, it is desirable for the natural-based agent to be water soluble.

In one embodiment, the natural-based finish composition is a surfactant. As used herein the term “surfactant” means an amphiphile (a molecule possessing both polar and nonpolar regions which are covalently bound) capable of reducing the surface tension of water and/or the interfacial tension between water and an immiscible liquid.

In some embodiments, the natural-based finish composition comprises a solution. In other embodiments, the composition may comprise a dispersion.

In certain embodiments, an aqueous solution comprising the natural-based finish composition may be prepared by heating a solution comprising the natural-based agent in water prior to application to the nonwoven fabric. Typically, the solution is heated to a temperature range that is from about 60 to 80° C., and the agents and solution are mixed under agitation for sufficient time to form a homogenous solution.

In certain embodiments, it has been found that cooling the thus formed solution containing the natural-based finish composition prior to application to the nonwoven fabric will improve the amount of the natural-based finish composition that is adhered to the fibers of the nonwoven fabric. In one embodiment, the heated solution is passed through a heat exchanger to obtain a solution temperature that is from about 25 to 30° C.

In certain embodiments, the amount of the dried natural-based finish composition present on the nonwoven fabric is from about 0.2 to 2 weight percent, based on the total weight of the nonwoven fabric. In some embodiments, the amount of the natural-based finish composition is from about 0.4 to 1.5 weight percent, based on the total weight of the nonwoven fabric, and in particular from about 0.5 to 1.0 weight percent, and more particularly, from about 0.5 to 0.8 weight percent, based on the total weight of the nonwoven fabric.

Optional Components

In some embodiments, it may also be useful to optionally treat the nonwoven fabric with finishes containing functional additives or other chemicals, such as antimicrobial agents, flame retardant agents, catalysts, lubricants, softeners, light stabilizers, antioxidants, colorants such as dyes and/or pigments, antistatic agents, fillers, odor control agents, perfumes and fragrances, and the like, and combinations thereof. Other optional components may be included in the compositions described herein.

II. Bio-Based Nonwoven Fabric

In further aspects of the invention, bio-based nonwoven fabrics having improved fluid management properties are provided. In particular, embodiments of the present invention are directed to a nonwoven fabric having a high bio-based material content. Preferably, bio-based nonwoven fabrics in accordance with certain embodiments of the present invention have a bio-based material content of at least 90 weight % of the absorbent article, such as comprising a bio-based material content that is at least 91%, 92%, 93%, 94%, 95%, 96%, 97%, 98%, 99% or 100% by weight of the nonwoven fabric.

In some embodiments, the bio-based material may comprise bio-based or biodegradable polymer materials. “Biodegradable” refers to a material or product which degrades or decomposes under environmental conditions that include the action of microorganisms. Thus a material is considered as biodegradable if a specified reduction of tensile strength and/or of peak elongation of the material or other critical physical or mechanical property is observed after exposure to a defined biological environment for a defined time. Depending on the defined biological conditions, a product comprised of a bio-based material might or might not be considered biodegradable.

A special class of biodegradable product made with a bio-based material might be considered as compostable if it can be degraded in a composing environment. The European standard EN 13432, “Proof of Compostability of Plastic Products” may be used to determine if a fabric or film comprised of sustainable content could be classified as compostable.

Preferably, bio-based nonwoven fabrics in accordance with embodiments of the present invention are substantially free of synthetic materials, such as petroleum-based materials and polymers. For example, bio-based nonwoven fabrics in accordance with certain embodiments of the present invention have less than 10 weight percent of materials that are non-bio-based, and more preferably, less than 5 weight percent, less than 4 weight percent, less than 3 weight percent, and even more preferably, less than 1 weight percent of non-bio-based materials, based on the total weight of the bio-based nonwoven fabric

Preferably, the bio-based nonwoven fabric comprises bio-based polymers that are sourced from a biological source Examples of bio-based polymers that may be used in certain aspects of the invention include the aliphatic polyesters previously described above.

Additional nonlimiting examples of bio-based polymers include polymers directly produced from organisms, such as polyhydroxyalkanoates (e.g., poly(beta-hydroxyalkanoate), poly(3-hydroxybutyrate-co-3-hydroxyvalerate, NODAX™), and bacterial cellulose, polymers extracted from plants and biomass, such as polysaccharides and derivatives thereof (e.g., gums, cellulose, cellulose esters, chitin, chitosan, starch, chemically modified starch), proteins (e.g., zein, whey, gluten, collagen), lipids, lignins, and natural rubber; and current polymers derived from naturally sourced monomers and derivatives, such as bio-polyethylene, bio-polypropylene, polytrimethylene terephthalate, polylactic acid, NYLON 1 1, alkyd resins, succinic acid-based polyesters, and bio-polyethylene terephthalate.

In some embodiments, the bio-based polymer may comprise bio-based polyethylene that is derived from a biological source. For example, bio-based polyethylene can be prepared from sugars that are fermented to produce ethanol, which in turn is dehydrated to provide ethylene. An example of a suitable sugar cane derived polyethylene is available from Braskem S.A. under the product name PE SHA7260.

Bio-based nonwoven fabrics in accordance with certain embodiments may include spunbond fabrics, meltblown fabrics, airlaid fabrics, wet-laid fabrics, coformed fabrics, bi-formed fabrics, carded fabrics and the like. In a preferred embodiment, the bio-based nonwoven fabric comprises a spunbonded fabric.

Bio-based nonwoven fabrics in accordance with the invention may comprise monocomponent, bicomponent, or multicomponent fibers. Examples of bicomponent fibers include side-by-side, islands in the sea, and sheath/core arrangements. Preferably, the fibers have a sheath/core structure in which the sheath comprises a first polymer component, and the core comprises a second polymer component. In this arrangement, the polymers of the first and second polymer components may be the same or different from each other.

In some embodiments, the bio-based nonwoven comprises a bi-form configuration in which PLA meltblown filaments are mechanically integrated with natural fibers, such as cotton fibers, wood pulp fibers, rayon fibers, animal fibers, hemp fibers, and the like. In some embodiments, cotton linters may be used in the process depending on the desired fluid management properties.

In certain embodiments, the bio-based nonwoven fabrics comprise monocomponent filaments comprising an aliphatic polyester described above, or a blend of such aliphatic polyesters. In other embodiments, the bio-based nonwoven fabric may comprise multicomponent filaments in which the aliphatic polyester component defines at least a portion of the exterior surfaces of the fibers.

In a preferred embodiment, the fibers of the nonwoven fabric have a bicomponent arrangement in which the aliphatic polyester comprises a first polymer component defining a sheath, and a second bio-based polymer component comprises the core.

In preferred embodiments, the sheath and the core both comprise an aliphatic polyester resin.

In certain preferred embodiments, the aliphatic polyester comprises PLA. In these embodiments, a PLA spunbond nonwoven fabric may be provided that is substantially free of synthetic polymer components, such as petroleum-based materials and polymers. For example, the fibers of the PLA spunbond nonwoven fabric may have a bicomponent arrangement in which both components are PLA based to thus produce a fiber that is 100% PLA.

As used herein, “100% PLA” may also include up to 5% additives including additives and/or masterbatches of additives to provide, by way of example only, color, softness, slip, antistatic protection, lubricity, hydrophilicity, liquid repellency, antioxidant protection and the like. In this regard, the nonwoven fabric may comprise 95-100% PLA, such as from 96-100% PLA, 97-100% PLA, 98-100% PLA, 99-100% PLA, etc. When such additives are added as a masterbatch, for instance, the masterbatch carrier may primarily comprise PLA in order to facilitate processing and to maximize sustainable content within the fibers.

For example, the PLA spunbond nonwoven fabric layer may comprise one or more additional additives. In such embodiments, for instance, the additive may comprise at least one of a colorant, a softening agent, a slip agent, an antistatic agent, a lubricant, a hydrophilic agent, a liquid repellent, an antioxidant, and the like, or any combination thereof.

In one embodiment, the PLA polymer of the sheath may be the same PLA polymer as that of the core. In other embodiments, the PLA polymer of the sheath may be a different PLA polymer than that of the core. For example, the bicomponent fibers may comprise PLA/PLA reverse bicomponent fibers such that the sheath comprises a first PLA grade, the core comprises a second PLA grade, and the first PLA grade and the second PLA grade are different (e.g., the first PLA grade has a higher melting point than the second PLA grade). By way of example only, the first PLA grade may comprise up to about 5% crystallinity, and the second PLA grade may comprise from about 40% to about 50% crystallinity.

In other embodiments, for instance, the first PLA grade may comprise a melting point from about 125° C. to about 135° C., and the second PLA grade may comprise a melting point from about 155° C. to about 170° C. In further embodiments, for example, the first PLA grade may comprise a weight percent of D isomer from about 4 wt. % to about 10 wt. %, and the second PLA grade may comprise a weight percent of D isomer of about 2 wt. %.

For example, in one embodiment, the core may comprise a PLA having a lower % D isomer of polylactic acid than that of the % D isomer PLA polymer used in the sheath. The PLA polymer with lower % D isomer will show higher degree of stress induced crystallization during spinning while the PLA polymer with higher D % isomer will retain a more amorphous state during spinning. The more amorphous sheath will promote bonding while the core showing a higher degree of crystallization will provide strength to the fiber and thus to the final bonded web. In one particular embodiment, the Nature Works PLA Grade PLA 6752 with 4% D Isomer can be used as the sheath while NatureWorks Grade 6202 with 2% D Isomer can be used as the core.

In other embodiments of the bio-based nonwoven fabric, the fibers may comprise multicomponent filaments in which PLA comprises one of the components and a different aliphatic polymer comprises a second polymer component. For example, in one embodiment, the multicomponent fabric may comprise a first polymer component comprising a PLA resin, and a second polymer component comprising a PBS resin. In one such embodiment, the bio-based nonwoven fabric may have a core comprising PLA and a sheath comprising PBS. In some embodiments, both the core and sheath may comprise a PBS resin.

In other embodiments, bio-based nonwoven fabrics may have a sheath/core configuration in which the aliphatic polyester component comprises the sheath, and a bio-based polyethylene component comprises the core. As discussed previously, the aliphatic polyester helps to adhere the natural-based finish composition to the surface of the fibers.

Fabric Properties

Fabrics in accordance with certain embodiments of the invention exhibit improved hydrophilicity in comparison to the identical fabric that has not been treated with the natural-based finish composition. Generally, water droplet contact angle is an indication of the hydrophobic/hydrophilic nature of a material. In one embodiment of the invention, the inventive nonwoven fabric exhibits a water droplet contact angle that is less than 125°, such as less than 120°, or less than 115°, and in particular less than 110°, and more particularly, less than 105°. In certain embodiments, inventive nonwoven fabrics in accordance with embodiments of the invention exhibit a water droplet contact angle that is from about 80° to 125°, such as from about 85° to 120° or 90° to 115°. In some embodiments, inventive nonwoven fabrics exhibit a water droplet contact angle that is from about 85° to 115°, and more typically, from about 90° to 110°, and more particularly, from about 95° to 105°. In one embodiment, the nonwoven fabric exhibits a water droplet contact angle that is from 98° to 102°.

Nonwoven fabrics in accordance with the invention may exhibit a water droplet contact angle that is less than 125°, less than 124°, less than 123°, less than 122°, less than 121°, less than 120°, less than 119°, less than 118°, less than 117°, less than 116°, less than 115°, less than 114°, less than 113°, less than 112°, less than 111°, less than 110°, less than 109°, less than 108°, less than 107°, less than 106°, less than 105°, less than 104°, less than 103°, less than 102°, less than 101°, less than 100°, less than 99°, less than 98°, less than 97°, less than 96°, less than 95°, less than 94°, less than 93°, less than 92°, less than 91°, and less than 90°.

In certain embodiments, treated surfaces of nonwoven fabrics in accordance with embodiments of the present invention may exhibit a decrease in water droplet contact angle that is from 5 to 25% in comparison to identical nonwoven fabrics to which no natural-based finish composition has been applied. In one embodiment, the nonwoven fabric exhibits a decrease in water droplet contact angle that is at least 10% in comparison to an identical nonwoven fabrics to which no natural-based finish composition has been applied. In still another embodiment, the nonwoven fabric exhibits a decrease in water droplet contact angle that is at least 15% in comparison to an identical nonwoven fabric to which no natural-based finish composition has been applied. In a preferred embodiment, the nonwoven fabric exhibits a decrease in water droplet contact angle that is at least 20% in comparison to an identical nonwoven fabric to which no natural-based finish composition has been applied.

Advantageously, nonwoven fabrics in accordance with certain embodiments of the invention exhibit improved fluid management properties. In the context of the invention, fluid management refers generally to the fabric's ability to quickly transport fluid through the thickness of the fabric in one direction while at the same time preventing or retarding the flow of fluid back through the fabric in the opposite direction. In determining a fabric's fluid management properties, three tests are often utilized; fluid strikethrough, rewet, and run-off.

Fluid strikethrough measures the speed at which an insult of fluid on the surface of the fabric is removed. Typically, strikethrough is measured in a series of successive fluid insults on the surface of the fabric. Shorter strike through times are indicative of the fabric's ability to quickly move fluid away from the skin of the wearer and into an absorbent core.

Rewet measures the capacity of a nonwoven fabric to hold back fluids under pressure. Low rewet values are indicative of the fabric's ability to prevent a fluid from being transported back through the fabric where it could contact the skin of the wearer.

Run-off measures the amount of test fluid which runs down a nonwoven fabric test specimen when a specified mass of test liquid is poured on the nonwoven test specimen superimposed on a standard absorbent medium and placed on an inclined plane. Liquid that runs off the surface of the fabric is collected and weighed. In general, run-off values are indicative of the volume of fluid that can be absorbed through the fabric and into the absorbent medium. Low run-off values mean that most of the fluid is absorbed into the medium and not left in contact with the skin of the wearer.

Certain embodiments of the invention exhibit a good balance in fluid management properties as evidenced by strikethrough, rewet, and run-off values. In some embodiments, nonwoven fabrics in accordance with embodiments of the invention exhibit average fluid strikethrough times of less than 1.75 seconds after a first strike, less than 2.1 seconds after a second strike, and less than 2.2 seconds after a third strike.

In certain embodiments, nonwoven fabrics in accordance with the invention may exhibit a fluid rewet value of less than 1 gram (g), less than 0.5 g, less than 0.2 g, and in particular less than 0.175 g, and more particularly, less than 0.160 g.

In some embodiments, nonwoven fabrics in accordance with the invention may exhibit fluid run-off values of less than 1%, and in particular, less than 0.5%.

In one particular embodiment, a treated nonwoven fabric according to one of the preceding claims, wherein a treated nonwoven fabric exhibits a fluid strikethrough after a first insult of less than 5 seconds, a rewet of less than 1 gram; and a run-off of less than 5%.

In a further embodiment a treated nonwoven fabric exhibits a fluid strikethrough after a first insult of less than 2 seconds, a rewet of less than 0.5 grams; and fluid a run-off of less than 1%. In still yet a further embodiment, the treated nonwoven fabric exhibits the following a fluid strikethrough after a first insult of less than 1.75 seconds, a rewet of less than 0.2 grams; and a fluid run-off of less than 0.5%.

According to certain embodiments, for instance, the fabric may comprise a machine direction (MD) tensile strength at max from about 20 N/5 cm to about 75 N/5 cm. In other embodiments, for example, the fabric may comprise a MD tensile strength at max from about 22 N/5 cm to about 65 N/5 cm. In further embodiments, for instance, the fabric may comprise a MD tensile strength at max from about 50 N/5 cm to about 65 N/5 cm. As such, in certain embodiments the fabric may comprise a MD tensile strength at max from at least about any of the following: 20, 25, 26, 27, 28, 29, 30, 50, 60, 70, and 80 N/5 cm, and/or at most about 100, 75, 70, 65, 60, 55, 50, and 45 N/5 cm, (e.g., about 25-100 N/5 cm, about 30-75 N/5 cm, about 45 to 65 N/5 cm, etc.).

In certain embodiments, for example, the fabric may comprise a cross machine direction (CD) tensile strength at max from about 5 N/5 cm to about 85 N/5 cm. In other embodiments, for instance, the fabric may comprise a CD tensile strength at max from about 6 N/5 cm to about 75 N/5 cm. In some embodiments, for example, the fabric may comprise a CD tensile strength at max from about 7 N/5 cm to about 25 N/5 cm. As such, in certain embodiments, the fabric may comprise a CD tensile strength at max from at least about any of the following: 6, 7, 8, 9, 10, 11, 12, 13, 14, 15, 16, 17, 18, 19, 20, 21, 22, 23, 24, 25, 26, 27, 28, 29, and 30 N/5 cm and/or at most about 85, 80, 75, 70, 65, 60, 55, 50, 45, 40, 35, 30, 29, 28, 27, 26, and 25 N/5 cm (e.g., about 5-85 N/5 cm, about 8-30 N/5 cm, etc.).

In accordance with certain embodiments, for example, the nonwoven fabric may have a basis weight from about 7 grams per square meter (gsm) to about 150 gsm. In other embodiments, for instance, the fabric may have a basis weight from about 8 gsm to about 70 gsm. In certain embodiments, for example, the fabric may comprise a basis weight from about 10 gsm to about 50 gsm. In further embodiments, for instance, the fabric may have a basis weight from about 11 gsm to about 30 gsm. In one embodiment, the fabric may have a basis weight from about 15 gsm to about 25 gsm. As such, in certain embodiments, the fabric may have a basis weight from at least about any of the following: 7, 8, 9, 10, and 11 gsm and/or at most about 150, 100, 70, 60, 50, 40, and 30 gsm (e.g., about 9-60 gsm, about 11-40 gsm, etc.).

According to certain embodiments, for example, the fibers may have a linear mass density from about 0.6 dtex to about 3 dtex. In other embodiments, for instance, the fibers may have a dtex from about 1 dtex to about 2 dtex. In further embodiments, for example, the fibers may have a linear mass density from about 1.2 dtex to about 1.8 dtex. As such, in certain embodiments, the fibers have a linear mass density from at least about any of the following: 0.6, 0.7, 0.8, 0.9, 1.0, 1, 1.1, 1.2, 1.3, 1.4, 1.5, and 1.6 dtex and/or at most about 2.5, 2.4, 2.3, 2.2, 2.1, 2.0, 1.9, 1.8, and 1.7 dtex (e.g., about 1-2.5 dtex, about 1.1-1.8 dtex, etc.).

System and Method for Preparing the Nonwoven Fabric

Certain embodiments according to the invention provide systems and methods for preparing a nonwoven fabric having a natural-based finish composition disposed on a surface thereof.

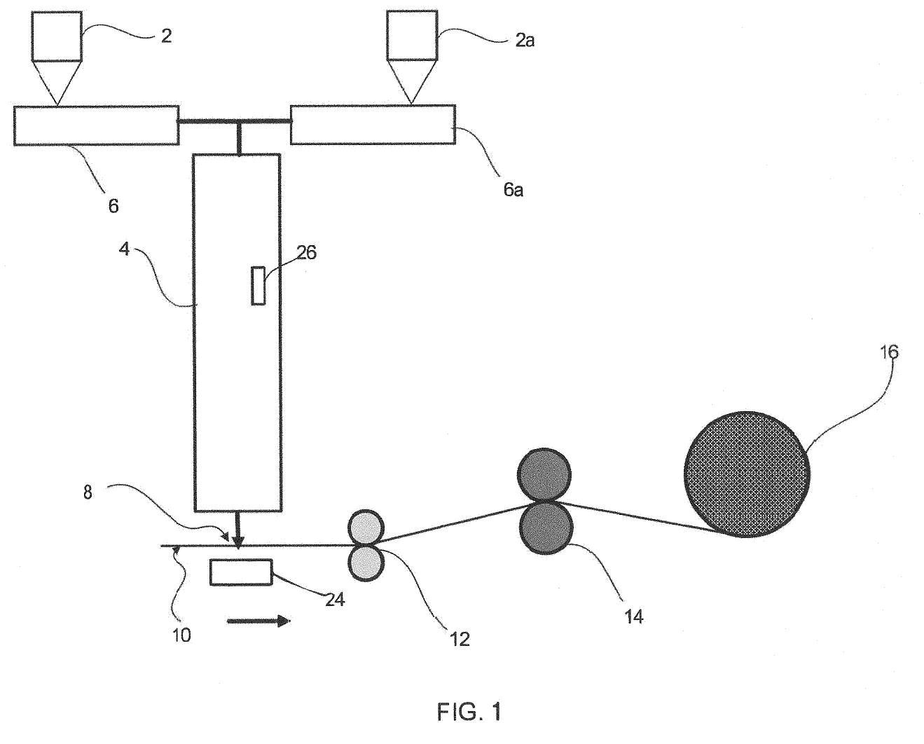

With reference to FIG. 1, for example, a schematic diagram of the a spunbond nonwoven fabric preparation system in accordance with certain embodiments of the invention is illustrated.

As shown in FIG. 1, a first polymer source (i.e. hopper) 2 is in fluid communication with the spin beam 4 via the extruder 6. A second polymer source (i.e. hopper) 2a is also in fluid communication with the spin beam 4 via extruder 6a. In the preparation of multicomponent fabrics, first polymer source may provide a stream of an aliphatic polyester resin, such as PLA, and the second polymer source may provide a stream of a polyolefin resin, such as polypropylene.

Following extrusion, the extruded polymer streams are introduced into the spin beam at which point the streams enter one or more spinnerets (not shown) for spinning into multicomponent filaments. Following spinning, the spun filaments may then be drawn (i.e. attenuated) via a drawing unit (not shown) and randomized in a diffuser. The spin beam 4 produces a curtain of filaments that is deposited on the collection surface 10 at point 8.

In one embodiment, the thus deposited filaments may then be bonded to form a coherent web. In some embodiments, an optional pair of cooperating rolls 12 (also referred to herein as a “press roll”) stabilize the web of the multicomponent continuous filaments by compressing the web before delivery to the calender 14 for bonding. In some embodiments, for example, the press roll may include a ceramic coating deposited on a surface thereof. In certain embodiments, for instance, one roll of the pair of cooperating rolls 12 may be positioned above the collection surface 10, and a second roll of the pair of cooperating rolls 12 may be positioned below the collection surface 10. The bonded spunbond nonwoven fabric moves to a winder 16, where the fabric is wound onto rolls.

In one embodiment, an optional humidity unit 26 may be used in conjunction with the spin beam 4 and/or the press roll 12 to reduce static via added moisture.

In accordance with certain embodiments, conventional spunbond processes may be used to prepare the inventive multicomponent nonwoven fabrics. In such embodiments, the process may include providing a stream of molten or semi-molten polypropylene resin, providing a stream of molten or semi-molten aliphatic polyester resin (e.g., PLA); combining the streams in a spin beam where the spin beam is configured to form multicomponent fibers in which the polypropylene and the aliphatic polyester define distinct components in the cross section of the fiber; forming a plurality of drawn multicomponent continuous filaments, depositing the plurality of multicomponent continuous filaments onto a collection surface, and bonding the plurality of multicomponent continuous filaments to form the spunbond nonwoven fabric. According to certain embodiments, for example, forming the plurality of multicomponent continuous filaments may comprise spinning the plurality of multicomponent continuous filaments, drawing the plurality of multicomponent continuous filaments, and randomizing the plurality of multicomponent continuous filaments.

In a preferred embodiment, the multicomponent filaments have a sheath/core configuration in which the aliphatic polyester component defines a sheath, and the polypropylene component defines the core.

In this regard, the spunbond nonwoven web may be produced, for example, by the conventional spunbond process wherein molten polymer is extruded into continuous filaments which are subsequently quenched, attenuated or drawn mechanically by draw rolls or pneumatically by a high velocity fluid, and collected in random arrangement on a collecting surface. After filament collection, any thermal, chemical or mechanical bonding treatment may be used to form a bonded web such that a coherent web structure results.

In accordance with certain embodiments, for instance, bonding the web to form the multicomponent spunbond nonwoven fabric may comprise thermal point bonding the web with heat and pressure via a calender having a pair of cooperating rolls including a patterned roll. In such embodiments, for example, thermal point bonding the web may comprise imparting a three-dimensional geometric bonding pattern onto the spunbond nonwoven fabric. The patterned roll may comprise a three-dimensional geometric bonding pattern. In some embodiments, for example, the bonding pattern at least one of a diamond pattern, a hexagonal dot pattern, an oval-elliptic pattern, a rod-shaped pattern, a CD rod-shaped pattern, or any combination thereof. In some embodiments, the calender may include a release coating to minimize deposit of molten or semi molten polymer on the surface of one or more of the rolls. As an example, such release coating is described in European Patent Application No. 1,432,860, which is incorporated herein in its entirety by reference.

In certain embodiments, the bonded area of the surface of the nonwoven fabric is in the range of about 8 to 40%, based on the total surface area of the side of the nonwoven fabric on which bonding occurs. In other embodiments, the bonded area is from about 10 to 32%, and in particular 12 to 20%, based on the total surface area of the nonwoven fabric.

In accordance with certain embodiments, for instance, the system may be configured to prepare the PLA continuous fibers at a fiber draw speed greater than about 2500 m/min. In other embodiments, for example, the system may be configured to prepare the PLA continuous fibers at a fiber draw speed from about 3000 m/min to about 4000 m/min. In further embodiments, for instance, the system may be configured to prepare the PLA continuous fibers at a fiber draw speed from about 3000 m/min to about 5500 m/min. As such, in certain embodiments, the system may be configured to prepare the PLA continuous fibers at a fiber draw speed from at least about any of the following: 2501, 2550, 2600, 2650, 2700, 2750, 2800, 2850, 2900, 2950, and 3000 m/min and/or at most about 5500, 4000, 3950, 3900, 3850, 3800, 3750, 3700, 3650, 3600, 3550, and 3500 m/min (e.g., about 2700-3800 m/min, about 3000-3700 m/min, etc.). Such speeds are merely exemplary, as the system may be run at fiber draw speeds slower than 2500 m/min as well. However, use of fiber draw speed significantly below 2500 m/min may begin to compromise fabric properties such as strength and resistance to shrinkage.

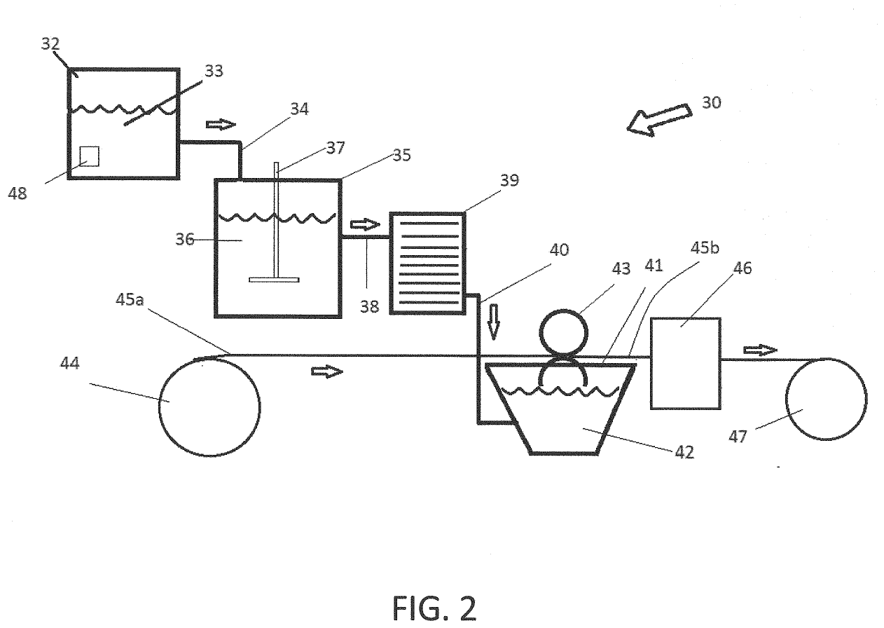

With reference to FIG. 2, a system for preparing and applying the natural-based finish composition to the nonwoven fabrics is shown and broadly designated by reference character 30. As shown, the system includes a source 32 of fluid (e.g., water) 33 that is heated via heater 48. The heater 48 is used to heat the fluid to a desired temperature. For example, in the preparation of a natural-based finish composition comprising milk or soy protein, the fluid may be heated to a temperature from about 60 to 90° C., with a temperature of about 70 to 80° C. being somewhat preferred.

The heated fluid is then transferred to a mixing tank 35 via fluid conduit 34. The heated fluid is then mixed with a natural-based finish agent under agitation via agitator 37 to produces a first stream of a heated natural-based finish composition. The natural-based finish agent may be metered into the fluid at a desired rate or added as one or more aliquots. In one embodiment, the agent and fluid are then mixed for a sufficient amount time to prepare a homogenous solution. The concentration of the natural-based finish agent in the resulting natural-based finish composition will generally depend on the agent used and the desired add-on concentration of the natural-based finish composition.

In certain embodiments, the concentration of the natural-based finish agent in the composition may range from about 50 to 80 g/L, and in particular, from about 60 to 70 g/L. In a preferred embodiment, the natural-based finish agent in the composition comprises a soy-based protein extract.

Additional agents, such as acids, bases, or pH buffers, may be introduced into the mixing tank 35. The heated stream of natural-based finish composition is then introduced into heat exchanger 39 via fluid conduit 38. In the heat exchanger 39, the natural-based finish composition is cooled to a temperature of less than 40° C., and in particular, a temperature ranging from about 25 to 35° C., with a temperature of about 30° C. being somewhat preferred. The now cooled solution is then introduced into application tank 41 as a stream of cooled composition comprising the natural-based agent. The cooled composition 42 is then applied to the surface of nonwoven fabric 45a via a kiss roll 43. The nonwoven fabric 45a is the inventive fabric discussed above which includes an aliphatic polyester component (e.g., PLA), and a polyolefin component (e.g., polypropylene). Nonwoven fabric may be supplied via supply roll 44.

Other methods of applying the natural-based finish composition to the nonwoven fabric may include gravure printing, immersion coating, padding, spraying, foam coating, or by any other method whereby one can apply a controlled amount of solution and/or suspension and/or dispersion comprising the natural-based finish composition to the surface of the nonwoven fabric.

After the composition containing the natural-based finish agent has been applied to the surface of the nonwoven fabric, the nonwoven fabric 45b is then passed through a dryer 46 (e.g., an oven or other source of heat) to remove the fluid from the nonwoven fabric. The nonwoven fabric 45b having the natural-based finish composition disposed thereon may then be collected on wind-up roll 47. In one embodiment, the treated nonwoven fabric may be dried at a temperature from about 70 to 110° C., and in particular, from about and 80 to 90° C.

Advantageously, embodiments of preparing the nonwoven fabric may done in a continuous process. In such embodiments, the composition comprising the natural-based finish agent may be continuously resupplied to the application tank 41 without needing to stop or slow down the process in order to prepare a new supply of the natural-based finish composition. In one such embodiment, the apparatus may include two or more separate mixing tanks in which batches of the natural-based finish composition is prepared. For example, each mixing tank may be in fluid communication with a source of heated fluid and a source of the natural-based agent. The sources of the heated fluid and the natural-based agent may be the same so that both mixing tanks are supplied from the same source, or they may be different sources.

Each separate tank of the heated composition comprising natural-based agent may be configured to provide a stream of heated composition as needed to the heat exchanger so that a continuous supply of the natural-based finish composition is maintained.

Nonwoven fabrics in accordance with embodiments of the present invention may be used in a wide variety of applications. In one embodiment, the nonwoven fabric may be combined with one or more additional layers to form a laminate. In particular, a laminate comprising the inventive nonwoven fabrics previously discussed may be adapted for use in a disposable absorbent article such as a diaper, a pant, an adult incontinence product, a sanitary napkin or any other article that may benefit from the desirable properties provided with the nonwoven fabrics in accordance with embodiments of the present invention.

A typical chassis of a disposable absorbent article may include a liquid pervious top sheet, a liquid impervious backsheet and an absorbent core disposed between the topsheet and the backsheet. An absorbent article may also include any features that may be suitable for such an article and are known in the art.

Nonwoven fabrics in accordance with embodiments of the invention may be used to prepare a variety of different structures. For example, in some embodiments, the inventive nonwoven fabric may be combined with one or more additional layers to prepare a composite or laminate material. Examples of such composites/laminates may include a spunbond composite, a spunbond-meltblown (SM) composite, a spunbond-meltblown-spunbond (SMS) composite, or a spunbond-meltblown-meltblown-spunbond (SMMS) composite. In some embodiments, composites may be prepared comprising a layer of the inventive nonwoven fabric and one or more film layers.

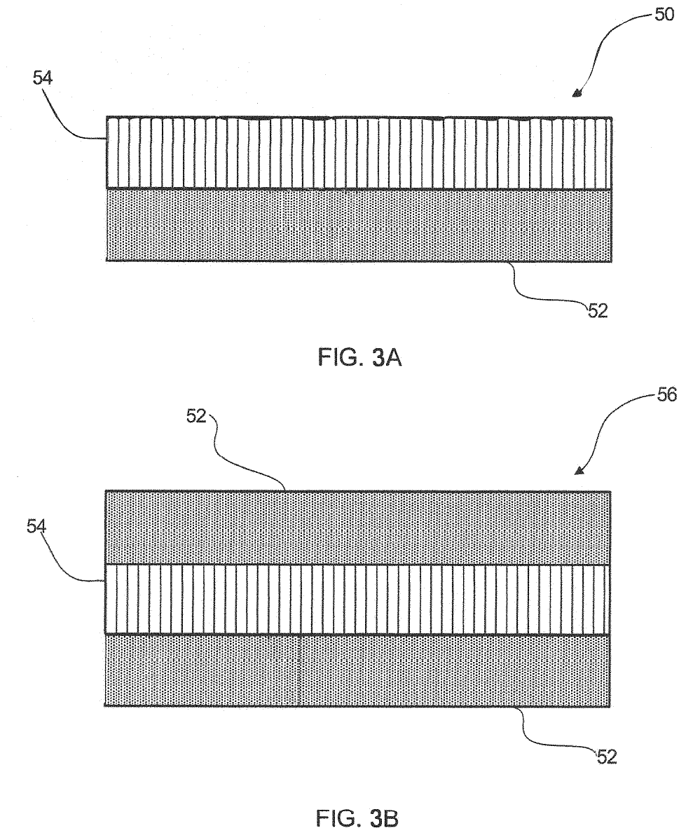

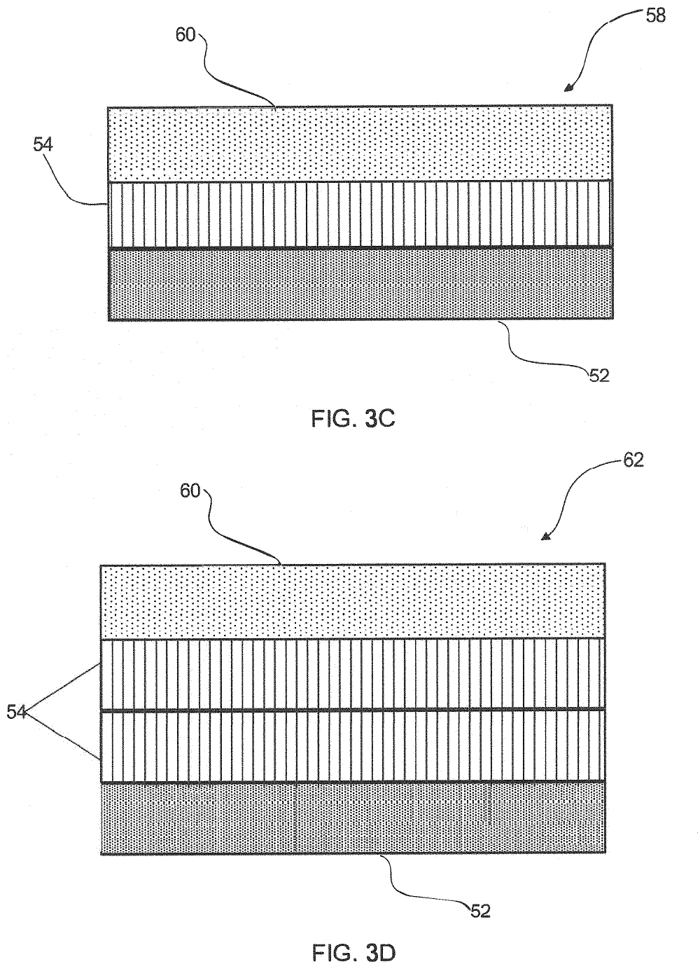

For example, FIGS. 3A-3D are cross-sectional views of composites in accordance with certain embodiments of the invention. For example, FIG. 3A illustrates a spunbond-meltblown (SM) composite 50 having a spunbond nonwoven fabric layer 52 in accordance with embodiments of the present invention, and a meltblown layer 54.

FIG. 3B illustrates a spunbond-meltblown-spunbond (SMS) composite 56 having two spunbond nonwoven fabric layers 52 and a meltblown layer 54 sandwiched between the spunbond nonwoven fabric layers 52.

FIG. 3C illustrates an SMS composite 58 having a spunbond nonwoven fabric layer 52, a different spunbond layer 60, and a meltblown layer 54 sandwiched between the two spunbond layers 52, 60.

Finally, FIG. 3D illustrates a spunbond-meltblown-meltblown-spunbond (SMMS) composite 62 having a spunbond nonwoven fabric layer 52, a different spunbond layer 60, and two meltblown layers 54 sandwiched between the two spunbond layers 52, 60. Although the SMMS composite 62 is shown as having two different spunbond layers 52 and 60, both spunbond layers may be the spunbond nonwoven fabric layer 52, which is in accordance with.

In these multilayer structures, the basis weight of the inventive spunbond nonwoven fabric layer may range from as low as 7 g/m² and up to 150 g/m². In such multilayered laminates, both the meltblown and spunbond fibers could have an aliphatic polyester on the surface to insure optimum bonding. In some embodiments in which the inventive spunbond layer is a part of a multilayer structure (e.g., SM, SMS, and SWIMS), the amount of the meltblown in the structure may range from about 1 to 30%, and in particular, from about 1 to 15% of the structure as a percentage of the structure as a whole.

Multilayer structures in accordance with embodiments can be prepared in a variety of manners including continuous in-line processes where each layer is prepared in successive order on the same line, or depositing a meltblown layer on a previously formed spunbond layer. The layers of the multilayer structure can be bonded together to form a multilayer composite sheet material using thermal bonding, mechanical bonding, adhesive bonding, hydroentangling, or combinations of these. In certain embodiments, the layers are thermally point bonded to each other by passing the multilayer structure through a pair of calender rolls. In some embodiments, the layers are thermally bonded via air through bonding.

As previously noted, fabrics prepared in accordance with embodiments of the invention may be used in wide variety of articles and applications. For instance, embodiments of the invention may be used for personal care applications, for example products for babycare (diapers, wipes), for femcare (pads, sanitary towels, tampons), for adult care (incontinence products), or for cosmetic applications (pads), agricultural applications, for example root wraps, seed bags, crop covers, industrial applications, for example work wear coveralls, airline pillows, automobile trunk liners, sound proofing, and household products, for example mattress coil covers and furniture scratch pads.

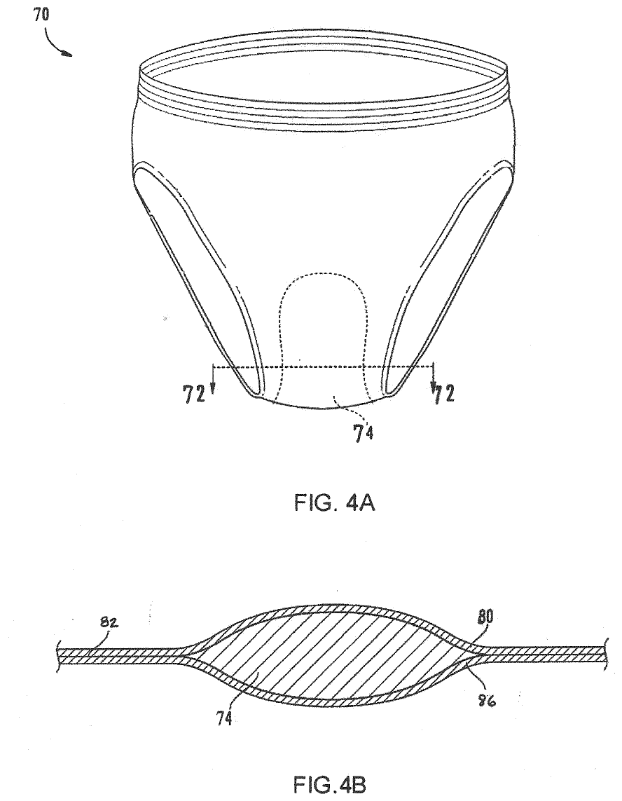

FIG. 4A, for example, is an illustration of an absorbent article (shown here as a diaper) in accordance with at least one embodiment of the invention and broadly designated by reference numeral 70. The diaper 70 may include an absorbent core 74. FIG. 4B is a cross-sectional view of the diaper 70 of FIG. 3A taken along line 72-72 of FIG. 4A. As shown in FIG. 4B, the absorbent core 74 may be sandwiched between a topsheet 80 and a backsheet 82. As further discussed herein, one or both of the topsheet 80 and the backsheet 82 may comprise a spunbond nonwoven fabric in accordance with embodiments of the invention as previously discussed in more detail herein.

The topsheet 80 is positioned adjacent an outer surface of the absorbent core 74 and is preferably joined thereto and to the backsheet 82 by attachment means (not shown) such as those well known in the art. For example, the topsheet 80 may be secured to the absorbent core 74 by a uniform continuous layer of adhesive, a patterned layer of adhesive, or an array of separate lines, spirals, or spots of adhesive.

As used herein, the term “joined” encompasses configurations whereby an element is directly secured to the other element by affixing the element directly to the other element, and configurations whereby the element is indirectly secured to the other element by affixing the element to intermediate member(s) which in turn are affixed to the other element. In a preferred embodiment of the present invention, the topsheet 80 and the backsheet 82 are joined directly to each other in the diaper periphery 86 and are indirectly joined together by directly joining them to the absorbent core 74 by the attachment means (not shown).

As discussed above, the topsheet is treated with a natural based finish composition to help ensure proper liquid transport through the topsheet and into the absorbent core.

In one embodiment, at least one of the topsheet and backsheet comprises a nonwoven fabric comprising the inventive nonwoven fabric as previously described herein.

There are a number of manufacturing techniques which may be used to manufacture the topsheet 80. For example, the topsheet 80 may be a nonwoven web of fibers. When the topsheet comprises a nonwoven web, the web may be spunbonded, carded, wet-laid, meltblown, hydroentangled, combinations of the above, or the like. A preferred topsheet comprises a spunbond nonwoven fabric in which the fibers are thermally bonded to each other to form a coherent web.

The backsheet 82 is positioned adjacent to an opposite surface of the absorbent core 74 and is preferably joined thereto by attachment mechanisms (not shown) such as those well known in the art. Suitable attachment mechanisms are described with respect to joining the topsheet 80 to the absorbent core 74. Alternatively, the attachment means may comprise heat bonds, pressure bonds, ultrasonic bonds, dynamic mechanical bonds, or any other suitable attachment means or combinations of these attachment mechanisms as are known in the art.

The backsheet 82 is impervious to liquids (e.g., urine) and is preferably manufactured from a thin plastic film, although other flexible liquid impervious materials may also be used. As used herein, the term “flexible” refers to materials which are compliant and will readily conform to the general shape and contours of the human body. The backsheet 82 prevents the exudates absorbed and contained in the absorbent core 74 from wetting articles which contact the diaper 70 such as bedsheets and undergarments. The backsheet 82 may thus comprise a woven or nonwoven material, polymeric films such as thermoplastic films, or composite materials such as a film-coated nonwoven material.

In some embodiments, material for the backsheet may include the inventive spunbond fabric described herein. In one embodiment, the backsheet may comprise a laminate structure having a liquid impervious film layer that is joined to a nonwoven fabric in accordance with one or more embodiments of the present invention. Suitable films may be prepared from polymers such as polyolefins including green polyethylene, polyesters, and bio-based polymers, such as polylactide based polymers. In one example, the film may comprise a sugar cane derived polyethylene polymer, such as a film grade LDPE polyethylene grade SEB853/72 or SPB681/59 recommended by Braskem S.A. for lamination. Suitable films may also include additives such as CaCO₃ to improve film breathability while still maintaining fluid barrier properties. In some embodiments, the backsheet layer may comprise a laminate structure having a film layer laminated to the inventive nonwoven fabric layer having a spunbond-meltblown-spunbond (SMS) structure.

The absorbent core 74 may comprise any material that is capable of absorbing fluids and exudates. In one embodiment, materials for the absorbent core may include a core wrap. The core wrap may comprise a fabric layer comprising a spunbond fabric, spunbond-meltblown fabric (SM), or an SMS fabric. An example of a core wrap comprising an SMS fabric comprises a spunbond nonwoven layer comprising bicomponent fibers having a sheath comprising the aliphatic polyester component, and core comprising the previously described polypropylene component.

In one embodiment, disposable absorbent articles may be prepared that include a back waist region, a crotch region and a front waist region. A pair of ears may be attached along their respective proximal edge to the left and right sides of the disposable absorbent article respectively. The disposable absorbent article may include a fastener such as a mechanical comprising a plurality of extending hooks or an adhesive may be connected to a portion of the ear or side panel about the distal edge of the ear or side panel. Such a fastener may in combination with the extensible laminate may provide for proper placement and attachment of the absorbent article about the lower torso of a wearer.

In another embodiment, any such extensible laminate may be used as an integral outer cover for an absorbent article. A typical chassis of a disposable absorbent article may include a liquid pervious top sheet, a liquid impervious backsheet and an absorbent core disposed between the topsheet and the backsheet. An absorbent article may also include any features that may be suitable for such an article and are known in the art.

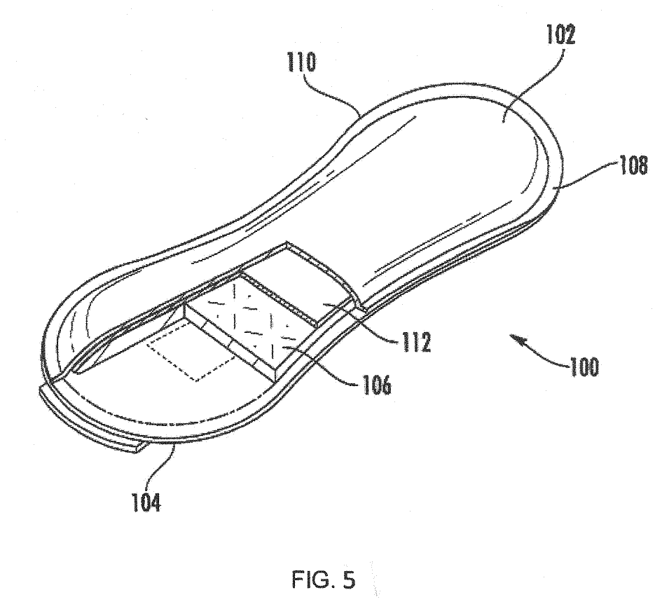

FIG. 5 is an illustration of an absorbent article in accordance with at least one embodiment of the invention in which the absorbent article is in the form of a feminine sanitary pad broad designated by reference numeral 100. Pad 100 may include a topsheet 102, backsheet 104, and an absorbent core 106 disposed there between. Preferably, topsheet 102 and backsheet 104 are joined to each other about along opposing outer edges to define a continuous seam 108 that extends about the periphery 110 of the pad 100. Continuous seam 108 may comprise a heat seal that is formed from thermally bonding the topsheet and backsheet to each other. In other embodiments, continuous seam 108 is formed by adhesively bonding the topsheet and backsheet to each other.

As in the embodiments discussed above, pad 100 preferably comprises a nonwoven fabric in accordance with the present invention. That is a spunbond nonwoven fabric comprising fibers having a polyethylene component, and a polypropylene component that is a blend of a first polypropylene polymer and a low isotacticity polypropylene polymer.

In some embodiments, pad 100 may also include a fluid acquisition layer 112 that is disposed between the absorbent core 106 and the topsheet 102.

Various components of the absorbent article are typically joined via thermal or adhesive bonding.

The following examples are provided for illustrating one or more embodiments of the present invention and should not be construed as limiting the invention.

### Examples

Spunbond nonwoven fabrics in the following examples were prepared with a Reicofil spunbond spinning lines produced by Reifenhaeuser. Unless otherwise indicated all percentages are weight percentages. The materials used in the examples are identified below.

Test Methods

Basis weight was measured in accordance with ASTM D-3776-09a.

Strike through was measured in accordance with WSP 70.3/ASTM 150.5.

Rewet was measured in accordance with WSP 80.10/ASTM 151.3.

Run-off was measured in accordance with NWSP 80.9.

Contact angle is measure in accordance with ASTM D 5946.

MD and CD tensile strengths and MD and CD elongations were determined in accordance with Test Method WSP 110.4 A with a sample width of 2 inches, crosshead speed of 12 inches per minute, and a gauge length of 3 inches.

In the following examples, nonwoven fabrics comprising bicomponent filaments were prepared and evaluated. The precursor nonwoven fabrics had basis weight of 15 gsm and 18 gsm, and comprised bicomponent filaments having a PLA sheath and a polypropylene core. The sheath/core ratio was 25:75. The PLA resin was obtained from NatureWorks, product number 6202D, and the polypropylene resin was obtained from Braskem, product number CP360H.

A solution containing a soy protein isolate (Pro-Fam® 781) was prepared in the following steps. Tap water was supplied to a heating tank and heated to a temperature of 80° C. 75 liters of heated water was then transferred to a mixing tank to which 4.8 kg of the soy protein was introduced into the tank. The water and soy protein were mixed under agitation for about 25 minutes and at a temperature of 80° C. The resulting solution was then passed through a heat exchanger to adjust the solution temperature to 30° C. Cooling the solution also resulted in a thickening of the solution.

The now cooled solution was introduced into a kiss roll bath and applied to one side (“the treated surface”) of the previously prepared nonwoven fabric at a line speed of 232 m/minute. The kiss roll was operated at a speed of 18 revolutions per minute. The nonwoven fabric was then dried to remove the water using an Omega through aid dryer. The dried fabric was wound on a winder roll. The average add-on weight of the dried soy protein was 0.7 weight percent, based on the total weight of the nonwoven fabric.

Hydrophilicity

Samples of the treated nonwoven fabric were prepared and the effects of the natural-based finish composition on hydrophilicity were evaluated by measuring the contact angle of the fabric. In Sample 1, the hydrophilicity of the nonwoven fabric was evaluated. Comparative Example 1 is the same nonwoven fabric (PLA/PP) prior to treatment with the natural-based finish composition. Comparative Example 2 is a nonwoven fabric comprising polypropylene monocomponent filaments with no finish composition applied.

For each sample, the contact angle was measured at two locations on the surface of the fabric using the tangent measurement method. A total of 10 measurements were taken. The averaged results are summarized in Table 1 below.

TABLE 1
Contact Angle

| | Average value | |
|---|---|---|
| | of contact angle | Standard |
| Example No. | (degrees) | Deviation |
| Example No. 1 | 99.8 | 8.5 |
| Comparative Example 1 | 129.1 | 8.3 |
| Comparative Example 2 | 143.2 | 7.7 |

From Table 1 above, Comparative Example 2 comprising polypropylene monocomponent filaments had an average contact angle of 143.2°. The untreated nonwoven fabric of Comparative Example 1 comprising PLA/PP bicomponent filaments had an average contact angle of 129.1°. In Example 1, the treated surface of the PLA/PP nonwoven fabric, had an average contact angle of 99.8°.

The results from Table 1 show that the untreated PLA/PP nonwoven fabric (Comparative Example 1) is slightly more hydrophilic than the nonwoven fabric comprised of polypropylene monocomponent filaments (Comparative Example 2).

Following application of the natural-based finish composition, the treated surface of the nonwoven fabric of Example 1 exhibited significant improvements with respect to hydrophilicity in comparison to the same fabric that has not been treated (Comparative Example 1) as evidenced by an average percent decrease in contact angle of 22.7%, and a percent difference of 25.6%.

Average physical properties of the treated 15 gsm nonwoven fabric are summarized in Table 2 below.

TABLE 2
Nonwoven Fabric Properties for 15 gsm PLA/PP Bicomponent

| Property | Average | N |
|---|---|---|
| Basis weight | 15.5 | 6 |
| (g/m²) | | |
| MD Tensile Peak | 24.4 | 6 |
| (N/5 cm) | | |
| MD Elongation at Peak | 17.83 | 6 |
| (%) | | |
| CD Tensile at Peak | 8.26 | 6 |
| (N/5 cm) | | |
| CD Elongation at Peak | 36.79 | 6 |
| (%) | | |
| Air Permeability | 790.83 | 6 |
| (CFM) | | |
| Denier | 1.69 | 1 |

The fluid management properties of the treated nonwoven fabrics and control samples were also evaluated. In this evaluation, fluid strikethrough, first rewet, and run-off for the surfaces of the 15 gsm and 18 gsm nonwoven fabrics, described above, were measured. The results are provided in Tables 3 and 5 below. Table 4 includes control samples in which fluid management properties of the 15 gsm PLA/PP nonwoven fabric were evaluated.

TABLE 3
Fluid Management Properties for Treated Nonwoven (15 gsm PLA/PP
Fabric)

| | | | | | Run-off |
|---|---|---|---|---|---|
| | Strike 1 | Strike 2 | Strike 3 | Rewet | (% of 25 mL |
| Sample No. | (s) | (s) | (s) | (g) | insult) |
| Sample 1 | 1.580 | 2.110 | 2.130 | 0.285 | 0.476 |
| Sample 2 | 1.510 | 2.150 | 2.170 | 0.154 | 0.344 |
| Sample 3 | 1.860 | 2.110 | 2.260 | 0.123 | 0.328 |
| Sample 4 | 2.210 | 2.340 | 2.450 | 0.141 | — |
| Sample 5 | 1.500 | 1.870 | 1.860 | 0.086 | — |
| Average | 1.732 | 2.116 | 2.174 | 0.158 | 0.383 |

TABLE 4
Fluid Management Properties for Control Sample (15 gsm PLA/PP
Fabric)

| | Strike 1 | Strike 2 | Strike 3 | Rewet |
|---|---|---|---|---|
| Sample No. | (s) | (s) | (s) | (g) |
| Control 1 | 3.300 | 3.880 | 3.420 | 0.075 |
| Control 2 | 3.940 | 4.550 | 3.740 | 0.082 |
| Control 3 | 3.870 | 4.6660 | 4.900 | 0.084 |
| Control 4 | 3.480 | 3.590 | 4.140 | 0.082 |
| Control 5 | 3.950 | 4.370 | 4.620 | 0.091 |
| Average | 3.708 | 4.210 | 4.164 | 0.083 |

TABLE 5
Fluid Management Properties for Treated Nonwoven (18 gsm PLA/PP
Fabric)

| | | | | | Run-off |
|---|---|---|---|---|---|
| | Strike 1 | Strike 2 | Strike 3 | Rewet | (% of 25 mL |
| Sample No. | (s) | (s) | (s) | (g) | insult) |
| Sample 6 | 1.530 | 2.050 | 1.980 | 0.719 | 0.432 |
| Sample 7 | 1.290 | 1.780 | 1.700 | 0.440 | 0.912 |
| Sample 8 | 1.610 | 2.030 | 2.130 | 0.143 | 1.172 |
| Sample 9 | 1.760 | 2.180 | 1.790 | 0.535 | — |
| Sample 10 | 1.570 | 2.040 | 2.170 | 0.309 | — |
| Average | 1.552 | 2.016 | 2.170 | 0.429 | 0.839 |

The strikethrough results for the treated nonwoven fabrics evaluated in Tables 3 and 5 are both good and showed slight increases in time with each successive insult. In addition, the inventive nonwoven fabrics (see Table 3) exhibited significant improvement in comparison to the untreated control fabrics. (see Table 4). The above date shows that the inventive nonwoven fabrics provide a good balance of fluid management properties. More specifically, while providing for rapid absorption of the water into the nonwoven fabric, rewet and run-off are minimized. In particular, the treated nonwoven fabric allowed for rapid strikethrough values while preventing/decreasing fluid rewet and run-off.

Accordingly, it is evident that nonwoven fabrics in accordance with certain embodiments of the invention exhibit a good balance between in removing fluid quickly through the fabric while prevent passage of fluid back through the fabric.

Modifications of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A nonwoven fabric comprising a plurality of fibers that are bonded to each other to form a coherent web, the fibers comprising an aliphatic polyester polymer defining at least a portion of an outer surface of the fibers, and wherein a natural-based finish composition is disposed on at least a portion of the outer surface of the fibers.

2. The nonwoven fabric of claim 1, wherein the fibers comprise multicomponent fibers having a bicomponent configuration, in which a first polymer component comprises the aliphatic polyester polymer and defines a sheath of the fibers, and a second polymer component comprises a polyolefin polymer defining a core of the fibers.

3. The nonwoven fabric according to claim 2, wherein the aliphatic polyester polymer comprises polylactic acid, and the polyolefin polymer comprises polypropylene polymer.

4. The nonwoven fabric of claim 1, wherein the fibers comprise a bio-based aliphatic polyester polymer.

5. The nonwoven fabric of claim 4, wherein the fibers have a sheath core configuration, and the bio-based aliphatic polyester polymer defines the sheath, and a second polymer component comprising a bio-based polymer is the core.

6. The nonwoven fabric according to claim 1, wherein the natural-based finish composition comprises a plant or animal based protein.

7. The nonwoven fabric according to claim 1, wherein the natural-based finish composition comprises a soy protein isolate.

8. The nonwoven fabric according to claim 1, wherein the natural-based finish composition is present in an amount ranging from about 0.2 to 2 weight percent, based on the total weight of the nonwoven fabric.

9. The nonwoven fabric according to claim 1, wherein the nonwoven fabric exhibits a water droplet contact angle of less than 110 degrees.

10. The nonwoven fabric according to claim 1, wherein the nonwoven fabric treated with the natural-based finish composition exhibits a decrease of at least 10% in contact angle in comparison to the identical nonwoven fabric that has not been treated with the natural-based composition.

11. The nonwoven fabric according to claim 1, wherein the nonwoven fabric exhibits the following:
a fluid strikethrough after a first insult of less than 5 seconds;
a rewet of less than 1 gram; and
a run-off of less than 5%.

12. An absorbent article comprising the nonwoven fabric of claim 1.

13. A composite sheet material comprising the nonwoven fabric of claim 1.

14. The composite sheet material of claim 13, wherein the sheet material comprises a meltblown layer.

15. An apparatus for preparing a nonwoven fabric comprising:
a supply source of a nonwoven fabric, the nonwoven fabric comprising a plurality of fibers comprised of an aliphatic polyester polymer, the aliphatic polyester polymer being present on at least a portion of surfaces of the fibers;
a source of heated fluid;
a source of natural-based agent;
a mixing tank in fluid communication with the source of heated fluid and source of natural-based agent, the mixing tank configured and arranged to mix the heated fluid and natural-based agent to produce a stream of heated composition comprising the natural-based agent;
a heat exchanger in fluid communication for cooling the stream of heated composition comprising the natural-based agent to produce a stream of cooled composition comprising the natural-based agent;
an application tank in fluid communication with the heat exchanger, the application tank configured and arranged to apply the cooled composition comprising the natural-based agent to fibers of the nonwoven fabric; and
a dryer for removing fluid from the nonwoven fabric that has been treated with the composition comprising the natural-based agent.

16. A method for preparing a nonwoven fabric comprising:
providing a nonwoven fabric, the nonwoven fabric comprising a plurality of fibers comprised of an aliphatic polyester polymer, the aliphatic polyester polymer being present on at least a portion of surfaces of the fibers;
heating a fluid;
adding a natural-based agent to the heated fluid;
mixing the natural-based finish agent and heated fluid to produce a stream of heated composition comprising the natural-based agent;
cooling the heated composition comprising the natural-based agent to produce a stream of cooled composition comprising the natural-based agent;
applying the cooled composition comprising the natural-based agent to fibers of the nonwoven fabric; and
drying the nonwoven fabric that has been treated with the composition comprising the natural-based agent.

17. The method according to claim 16, wherein the cooled composition comprising the natural-based agent is applied to a surface of the nonwoven fabric via a kiss roll.

18. The method according to claim 16, further comprising continuously applying the composition comprising the natural-based agent to at least one surface of the nonwoven fabric.

19. The method according to claim 16, wherein the step of cooling the heated composition comprises cooling the composition to a temperature ranging from about 25 to 35° C.

20. The method according to claim 16, wherein two separate streams of heated composition comprising the natural-based agent are produced and successively introduced into a heat exchanger to provide a continuous stream of the cooled composition comprising the natural-based agent.
